(19) United States
(12) Patent Application Publication
(10) Pub. No.: US 2020/0131096 A1
(43) Pub. Date: Apr. 30, 2020

(54) Biofertilizer Composition and Method of Manufacture

(71) Applicant: Sustainable Community Development, LLC, Kansas City, MO (US)

(72) Inventors: Sabeshan Kanagalingam, Kansas City, MO (US); Daoxin Li, Independence, MO (US)

(21) Appl. No.: 16/667,677

(22) Filed: Oct. 29, 2019

Related U.S. Application Data
(60) Provisional application No. 62/752,123, filed on Oct. 29, 2018.

(51) Int. Cl. C05F 11/08; C05F 17/20

(52) U.S. Cl. CPC C05F 11/08; C12N 1/20; C05F 17/20

(57) ABSTRACT

A biofertilizer composition and method of manufacturing the same contains a microorganism consortium in culture medium, a first additive having an organic acid chelating agent, a second additive having a nitrogen source, a potassium source, a chelated metal, and a salt, and water. The microorganism consortium comprises, at least, beneficial lactic acid fermenting microbes, bacteria belonging to the Bacilli class, and yeast.

### CROSS-REFERENCE TO RELATED APPLICATION

This application claims priority to U.S. provisional patent application No. 62/752,123 filed in the United States Patent and Trademark Office on Oct. 29, 2018. Application No. 62/752,123 and any materials incorporated by reference therein are hereby incorporated by reference herein.

### TECHNICAL FIELD

The present disclosure relates to microbial compositions and methods of using them. In particular, the present disclosure relates to a microbial consortia composition useful as a biofertilizer, along with methods of manufacturing the same. In some embodiments, the composition comprises at least two bacterial species co-cultured with at least one yeast. The composition is capable of beneficial use in numerous industries including agriculture, wastewater remediation, animal feed and feed additives, household cleaning, and as a chemical replacement in manufacturing processes for leather, paper, and other goods.

### BACKGROUND

Scientific research and public opinion both increasingly value beneficial microorganisms and their specialized properties. Microorganisms play an important role in agriculture, animal health, human health, and waste management. In agriculture, microorganisms are used to fertilize plants, enhance composting, and amend soil. In animals and humans, beneficial bacteria are used to prevent illness caused by harmful bacteria invading the natural flora. In waste management, microorganisms are used to accelerate waste decomposition and degrade odorous compounds.

Within agriculture, the demand to replace or supplement traditional fertilizers with environmentally-friendly biofertilizers is increasing. However, biofertilizers face challenges related to stability, storage, and effectiveness. Accordingly, there is a need to develop microorganism-based technologies and products that are stable under various conditions, maintain high titers of microbes, have an appreciable shelf life, and can be easily used.

### SUMMARY

The present disclosure provides a biofertilizer composition comprising at least one microorganism consortium in culture medium, at least one first additive, at least one second additive, and water. In some embodiments, the first additive may comprise at least one organic acid chelating agent, the second additive may comprise a composition comprising one or more of an amino acid, a peptide, a chelated metal, and a salt, and the water may comprise dechorinated water.

In some embodiments, the first additive may comprise fulvic acid, water, and in some embodiments, other ingredients. In some embodiments, the first additive comprises up to 20% fulvic acid, up to 70% kelp or kelp-derived material, and up to 80% water.

In some embodiments, the second additive may comprise at least 5.0% (w/w) water soluble nitrogen, 8.0% (w/w) soluble potash, 0.6% (w/w) boron, 1.6% (w/w) iron, 1.6% (w/w) manganese, and 1.6% (w/w) zinc. Moreover, in some embodiments, the second additive may comprise at least 5.0% (w/w) protein hydrolysate, 8.0% (w/w) potassium citrate, 0.6% (w/w) disodium octaborate tetrahydrate, 1.6% (w/w) ferrous sulfate, 1.6% (w/w) manganese sulfate, and 1.6% (w/w) zinc sulfate.

In some embodiments, the water may comprise any water whether modified or unmodified. In some embodiments, the water may comprise chlorine-free water. Chlorine-free water (or “dechlorinated” water) may be obtained by exposing the water to air and allowing the chlorine to evaporate, by carbon-filtering the water, or any by other known means of dechlorinating water. In some embodiments, however, the water may comprise filtered water, twice filtered water, treated water, water having additives, filtered sewage, runoff water, greywater, tap water, well water, carbonated water, seawater, lakewater, pondwater, rainwater, any of which may be captured, redirected, modified, or produced by known means.

In an aspect, the microorganism consortium in culture medium may comprise a combination of at least five lactic acid bacteria including Bacillus subtilis, a Lactobacillus sp., a Bifidobacterium sp., a Lactococcus sp., a Streptococcus sp., Acetobacter ghanensis, Gluconacetobacter diazotrophicus, Saccharomyces cerevisiae; and at least one purple non-sulfur bacterium.

In an aspect, the microorganism consortium in culture medium may comprise Acetobacter ghanensis, Bacillus coagulans, Bifidobacterium bifidum, Bifidobacterium longum, Gluconacetobacter diazotrophicus, Lactobacillus acidophilus, Lactobacillus bulgaricus, Lactobacillus casei, Lactobacillus fermentum, Lactobacillus lactis, Lactobacillus plantarum, Lactobacillus parafarraginis, Lactobacillus rapi, Saccharomyces cerevisiae, Streptococcus thermophilus, and at least one purple non-sulfur bacterium.

In another embodiment, the subject biofertilizer composition is in dry form, obtained by lyophilizing, spray-drying, or the like, a liquid composition containing a microorganism consortium in culture medium, a first additive, a second additive, and water. In some embodiments, the microorganism consortium in culture medium may comprise about 20,000 to 50,000 cfu/mL each of i) Acetobacter ghanensis, ii) Bacillus coagulans, iii) Bifidobacterium bifidum, iv) Bifidobacterium longum, v) Gluconacetobacter diazotrophicus, vi) Lactobacillus acidophilus, vii) Lactobacillus bulgaricus, viii) Lactobacillus casei, ix) Lactobacillus fermentum, x) Lactobacillus lactis, xi) Lactobacillus plantarum, xii) Lactobacillus parafarraginis, xiii) Lactobacillus rapi, and xiv) Saccharomyces cerevisiae; and ≥50,000 cfu/mL of at least one purple non-sulfur bacterium.

In an aspect, the present disclosure provides a method of manufacturing the biofertilizer composition. In one embodiment, the method contains the steps of (a) obtaining a microorganism consortium in culture medium, including but not limited to live lactic acid bacteria, a non-pathogenic gram-positive Bacilli bacterium capable of fermentation and a yeast, according to any combination disclosed herein, (b) adding a first additive, (c) adding a second additive, (d) adding water, (e) agitating the mixture, and (f) extracting the product. In some embodiments, the method may further comprise the steps of (i) adding a carbon source to the mixture, (ii) adding an essential oil to the mixture, or (iii) concentrating (such as decanting) the mixture before extracting the product. Furthermore, the method may comprise one or more steps comprising fermenting the mixture.

Methods of using the biofertilizer composition include applying the biofertilizer composition to soil in liquid or dry form and applied to the soil by methods known in the art, such as but not limited to spraying, dropping, scattering, injection, and dusting the target soil, or applied to a water source that feeds the target soil and applied in conjunction with other macronutrients and micronutrients, mixed therein. Moreover, the biofertilizer composition may be applied directly to plants or wastewater, and in some embodiments, to silage.

### DETAILED DESCRIPTION

The present disclosure may comprise one or more compositions having a micobial consortium in culture medium, at least one first additive, at least one second additive, and water. In some embodiments, the first additive may comprise an organic acid chelating agent and the second additive may comprise a composition comprising one or more of a nitrogen source, a potassium source, a chelated metal, and a salt. Methods of manufacturing the one or more biofertilizer compositions of the present disclosure may comprise combining the microbial onsortium in culture medium, the first additive, the second additive, and water, proceeding to agitate the mixture for an hour, then extracting the resulting biofertilizer composition. In some embodiments, the method may additionally comprise the step of concentrating the mixture before extracting the biofertilizer composition.

### I. Compositions

Compositions useful in this invention include microorganisms and additives. The microorganisms may include species of bacteria and fungi, including yeast and mold species. Suitable microorganisms include those commonly known in the art as phosphorus-solubilizing, potassium-solubilizing, nitrogen-fixing (collectively, biofertilizer), phototrophic, lactic acid, and sulfide-utilizing microorganisms.

It is contemplated that where two or more microorganisms are present within one or more embodiments of the composition, the microorganisms may be co-cultured. The microorganisms may be propagated by methods known in the art. For example, the microorganisms may be propagated in a liquid medium under anaerobic or aerobic conditions. Suitable liquid mediums used for growing microorganisms include those known in the art. It is contemplated that, in some embodiments, the one or more strains of useful microorganisms described herein may be co-cultured under laboratory conditions in any combination before being transferred to one or more fermentation tanks. In some embodiments, the one or more strains of useful microorganisms may be co-cultured partly under laboratory conditions, then transferred to one or more fermentation tanks, where the co-culture process may continue. In other embodiments, the one or more strains of useful microorganisms may be co-cultured within one or more fermentation tanks.

In one embodiment, the microorganism consortium in culture media of the biofertilizer composition includes live microorganisms. Preferably, at least one microorganism is included in the microorganism consortium in culture medium of the invention. More preferably, the compositions include consortia of two or more microorganisms. In another embodiment, the microorganism consortium in culture media of the biofertilizer composition includes living and non-living microorganisms. In another embodiment, the biofertilizer composition includes living or non-living microorganisms. Compositions containing non-living microorganisms may contain extracts of the microorganisms. Such extracts may be considered a liquid fermentation product of the living microorganisms. The extracts of microorganisms include, by way of example, enzymes, metabolites, proteins, and other substances that are produced by microorganisms and are capable of eliciting an effect on an environment regardless of the living status and/or metabolic states of the microorganism.

In one embodiment, the biofertilizer composition may be fermented, and may therein to produce one or more fermentation products. The biofertilizer composition may be fermented for about 1, 2, 3, 4, 5, 6, 7, 8, 9, 10, 11, 12, 13, 14, 15, 16, 17, 18, 19, 20, 21, 22, 23, 24, 25, or more days. Preferably, the biofertilizer composition is fermented for at least about 15 to about 23 days. More preferably, the biofertilizer composition is fermented for at least 21 days. More preferably, the biofertilizer composition is fermented for at least 15 days.

In one embodiment, the process of fermentation may result in one or more fermentation products. The one or more fermentation products may comprise an organic acid, an alcohol, or another type of compound. In another embodiment, the one or more fermentation products may comprise fermented sugar cane molasses or fermented rice bran.

In one embodiment, the composition includes a total number of microorganisms of about 1 to about 1 million colony forming units (CFU) per milliliter. Preferably, the composition includes a total number of microorganisms of about 100,000 to about 800,000 CFU per milliliter. More preferably, the composition includes a total number of microorganisms of about 250,000 to about 600,000 CFU per milliliter. Most preferably, the composition includes a total number of microorganisms of about 300,000 CFU per milliliter.

In one embodiment, the biofertilizer composition contains a first mixture of live microorganisms in the at least one microorganism consortium in culture medium, wherein the live microorganisms are present at a collective “low titer”, and one or more additional non-pathogenic gram-positive bacteria of the Bacilli family that are capable of fermentation at a “high titer” for each additional individual species/strain of bacteria, and a fermenting yeast. By collective titer, what is meant is the individual titers of each of the first live microorganism adds up to the collective titer. To use an example of live lactic acid bacteria (LAB), if the (LAB) consists of 15,000 cfu/mL of a Bacillus sp., 12,000 cfu/mL of a Lactobacillus sp., and 10,000 cfu/mL of a Streptococcus sp., then the collective titer is 37,000 cfu/mL of LAB. In one embodiment, “low titer” is <300,000 cfu/mL, about 0.001-299,999 cfu/mL, about 1,000-250,000 cfu/mL, 5,000-200,000 cfu/mL, 10,000-150,000 cfu/mL, 15,000-100,000 cfu/mL, 20,000-75,000 cfu/mL, 25,000-60,000 cfu/mL, 30,000-55,000 cfu/mL, about 10,000 cfu/mL, about 11,000 cfu/mL, about 12,000 cfu/mL, about 13,000 cfu/mL, about 14,000 cfu/mL, about 15,000 cfu/mL, about 16,000 cfu/mL, about 17,000 cfu/mL, about 18,000 cfu/mL, about 19,000 cfu/mL, about 20,000 cfu/mL, about 21,000 cfu/mL, about 22,000 cfu/mL, about 23,000 cfu/mL, about 24,000 cfu/mL, about 25,000 cfu/mL, about 30,000 cfu/mL, about 35,000 cfu/mL, about 40,000 cfu/mL, about 45,000 cfu/mL, about 50,000 cfu/mL, about 55,000 cfu/mL, about 60,000 cfu/mL, about 65,000 cfu/mL, about 70,000 cfu/mL, about 75,000 cfu/mL, about 80,000 cfu/mL, about 85,000 cfu/mL, about 90,000 cfu/mL, about 95,000 cfu/mL, about 100,000 cfu/mL, about 105,000 cfu/mL, about 110,000 cfu/mL, about 115,000 cfu/mL, about 120,000 cfu/mL, about 125,000 cfu/mL, about 150,000 cfu/mL, about 175,000 cfu/mL, about 200,000 cfu/mL, about 225,000 cfu/mL, about 250,000 cfu/mL, about 275,000 cfu/mL, or about 299,000 cfu/mL.

In one embodiment, “high titer” is ≥300,000 cfu/mL, 300,000-10,000,000 cfu/mL, 500,000-1,000,000 cfu/mL, about 300,000 cfu/mL, about 325,000 cfu/mL, about 350,000 cfu/mL, about 375,000 cfu/mL, about 400,000 cfu/mL, about 425,000 cfu/mL, about 450,000 cfu/mL, about 475,000 cfu/mL, about 500,000 cfu/mL, about 525,000 cfu/mL, about 550,000 cfu/mL, about 575,000 cfu/mL, about 600,000 cfu/mL, about 625,000 cfu/mL, about 650,000 cfu/mL, about 675,000 cfu/mL, about 700,000 cfu/mL, about 725,000 cfu/mL, about 750,000 cfu/mL, about 775,000 cfu/mL, about 800,000 cfu/mL, about 825,000 cfu/mL, about 850,000 cfu/mL, about 875,000 cfu/mL, about 900,000 cfu/mL, about 925,000 cfu/mL, about 950,000 cfu/mL, about 975,000 cfu/mL, about 1,000,000 cfu/mL, about 1,250,000 cfu/mL, about 1,500,000 cfu/mL, about 1,750,000 cfu/mL, about 2,000,000 cfu/mL, about 2,500,000 cfu/mL, about 3,000,000 cfu/mL, about 3,500,000 cfu/mL, about 4,000,000 cfu/mL, about 4,500,000 cfu/mL, about 5,000,000 cfu/mL, about 5,500,000 cfu/mL, about 6,000,000 cfu/mL, about 6,500,000 cfu/mL, about 7,000,000 cfu/mL, about 7,500,000 cfu/mL, about 8,000,000 cfu/mL, about 8,500,000 cfu/mL, about 9,000,000 cfu/mL, about 9,500,000 cfu/mL, about 10,000,000 cfu/mL, about 1.5E+7, about 2E+7, about 2.5+7, about 3E+7, about 3.5E+7, about 4E+7, about 4.5E+7, about 5E+7, about 5.5E+7, about 6E+7, about 6.52E+7, about 7E+7, about 7.5E+7, about 8E+7, about 8.5E+7, about 9E+7, about 9.5E+7, about 1E+8, about 2E+8, about 3E+8, about 4E+8, about 5E+8, about 6E+8, about 7E+8, about 8E+8, about 9E+8, about 1E+9, about 3E+9, about 4E+9, about 5E+9, about 6E+9, about 7E+9, about 8E+9, about 9E+9, about 1E+10, about 2E+10, about 3E+10, about 4E+10, about 5E+10, about 6E+10, about 7E+10, about 8E+10, about 9E+10, or 1E+11 cfu/mL.

In those embodiments in which the biofertilizer composition is in a dry form, the liquid biofertilizer compositions described herein are dried. “Dry form” refers to a composition containing ≤15% water by weight, ≤14% water by weight, ≤13% water by weight, ≤12% water by weight, ≤11% water by weight, ≤10% water by weight, ≤9% water by weight, ≤8% water by weight, ≤7% water by weight, ≤6% water by weight, ≤5% water by weight, ≤4% water by weight, ≤3% water by weight, ≤2% water by weight, ≤1% water by weight, 1%-5% water by weight, 2%-6% water by weight, about 3% water by weight, about 4% water by weight, about 5% water by weight, about 6% water by weight, about 7% water by weight, or about 8% water by weight. In one embodiment, the dry form of the biofertilizer composition is produced by evaporation, spray-drying, lyophilization, or the like. In some embodiments, the dry biofertilizer composition is encapsulated or combined with an excipient to promote the stability and viability of the microbes over time and under varying temperature conditions. In some embodiments, the dry biofertilizer composition is divided into micron scale particles that are subsequently coated with biocompatible polymers, such as polyethylene glycol (PEG), chitin, dextrin, polylactic glycolic acid copolymer (PLGA), polylactic acid (PLA), polyglycolic acid (PGA), or the like.

In some embodiments, the microorganism consortium in culture medium of the present disclosure may comprise, but is not limited to comprising, Bacillis subtilis, Bacillus coagulans, Lactobacillus acidophilus, Lactobacillus bulgaricus, Lactobacillus casei, Lactobacillus fermentum, Lactobacillus lactis, Lactobacillus parafarraginis, Lactobacillus plantarum, Lactobacillus rapi, Bifidobacterium bifidum, Bifidobacterium longum, Streptococcus thermophilus, Rhodopseudomonas palustris, Rhodobacter sphaeroides, Acetobacter ghanensis, Gluconacetobater diazotrophicus, and Saccharomyces cerevisiae.

In some embodiments, the microorganism consortium in culture medium of the present invention may comprise at least three microorganisms selected from the group consisting of purple non-sulfur bacteria, chromatiaceae green sulfur bacteria, colorless sulfur bacteria, filamentous green bacteria, and combinations thereof, wherein at least two microorganisms are selected from the group consisting of Bacillus subtilis, Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus casei, Lactobacillus fermentum, Lactobacillus plantarum, and Saccharomyces cerevisiae.

Alternatively, in some embodiments, the microorganism consortium in culture medium may comprise at least three microorganisms selected from the group consisting of purple non-sulfur bacteria, chromatiaceae, green sulfur bacteria, colorless sulfur bacteria, filamentous green bacteria, and combinations thereof, and at least two microorganisms selected from the group consisting of Bacillus subtilis, Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus casei, Lactobacillus fermentum, Lactobacillus plantarum, Rhodopseudomonas palustris, Rhodobacter sphaeroides, and Saccharomyces cerevisiae.

Alternatively, in some embodiments, the microorganism consortium in culture medium may comprise at least three microorganisms selected from the group consisting of purple non-sulfur bacteria, chromatiaceae, green sulfur bacteria, colorless sulfur bacteria, filamentous green bacteria, and combinations thereof, and at least two microorganisms selected from the group consisting of Bacillus subtilis, Bifidobacterium animalis, Bifidobacterium bifidum, Bifidobacterium longum, Enterococcus lactis, Streptococcus thermophilus, Lactobacillus acidophilus, Lactobacillus bulgaricus, Lactobacillus casei, Lactobacillus fermentum, Lactobacillus plantarum, Rhodopseudomonas palustris, Rhodobacter sphaeroides, and Saccharomyces cerevisiae.

In one or more embodiments, the microorganism consortium in culture medium of the present disclosure may comprise Bacillus subtilis, a Lactobacillus sp., a Bifidobacterium sp., a Lactococcus sp., Streptococcus thermophilus, a purple non-sulfur bacteria sp., at least two non-pathogenic gram-positive Bacilli bacteria capable of fermentation, and a yeast. In some embodiments, at least one purple non-sulfur bacterium is Rhodopseudomonas palustris, Rhodobacter sphaeroides, or a combination thereof. In some embodiments, the at least one yeast is Saccharomyces cerevisiae.

In some embodiments, the microorganism consortium in culture medium may comprise Bacillus subtilis, a Lactobacillus sp., a Bifidobacterium sp., a Lactococcus sp., Streptococcus thermophilus, at least one purple non-sulfur bacteria sp., at least two non-pathogenic gram-positive Bacilli bacteria capable of fermentation, and at least one yeast, and wherein at least one of said at least two gram-positive Bacilli bacteria is selected from the group consisting of Bifidobacterium spp., Lactobacillus spp., Lactococcus spp., Carnobacterium spp., Streptococcus spp., and Bacillus spp.

Further examples of useful microorganisms are presented in detail below and elsewhere herein. It is contemplated that the present disclosure is not limited to any one combination, but may instead comprise any combination of any microorganism articulated or described herein, and in any amounts or titer. Examples of phototrophic, lactic acid, biofertilizer, Bacilli family, and sulfide-utilizing microorganisms are found, for example, in Bergey's Manual of Determinative Bacteriology and Bergey's Manual of Systematic Bacteriology. For example, sulfide-utilizing microorganisms include species of Purple Non-sulfur Bacteria, Chromatiaceae, Green Sulfur Bacteria, Colorless Sulfur Bacteria, and Filamentous Green Bacteria. Biofertilizer microorganisms may include Lactobacillus genus, Enterococcus genus, Bifidiobacterium genus, Bacillus genus, Pseudomonas genus, Sporolactobacillus genus, Micromonospora genus, Micrococcus genus, Rhodococcus genus, and E. coli. Phototrophic microorganisms may include Rhodopseudomonas, Rodobactor, and combinations thereof. For example, phototrophic microorganisms may include Rhodopseudomonas palustris, R. sphaeroides, Rhodospirillum centenum, R. photometricum, R. rubrum, Rhodopila globiformis, Rhodobacter sphaeroides, and combinations thereof. Lactic acid microorganisms may include Lactobacillus, Lactococcus and combinations thereof. For example, lactic acid microorganisms may include Lactobacillus casei, L. plantarum, L. acidophilus, L. fermentum, L. brevis, L. lactis, L. reuteri, L. bulgaricus, L. cellobiosus, L. curvatus, L. delbrukil, L. helbeticus, L. euterii, L. salivarius, L. rhamnosus, L. gaserli, L. jensenii, L. sporogenes, Lactococcus lactis, Streptococcus (Enterococcus) faecium, S. faecalis, S. cremoris, S. diacetylactis, S. intermedius, S. lactis, S. thermophilus, Pediococuss acidilactici, P. cerevisiae (damnosus), P. pentosaceus, P. acidilacticii, Leuconostoc mesenteroides, and combinations thereof. Bacilli microorganisms may include Bacillus genus and combinations thereof. For example, Bacilli microorganisms may include Bacillus licheniformis, B. subtilus, B. toyoi, B. amyloliquefaciens, B. megateriu, B. pumilus, B. coagulans, B. lentus, B. thermophilus, B. laterosporus, B. cereus, B. circulans, and combinations thereof. Bifidobacterium microorganisms may include Bifidobacterium genus and combinations thereof. For example, Bifidobacterium microorganisms may include Bifidobacterium bifidum, B. pseudolongum, B. thermophilus, B. adolescentis, B. animalis, B. infantis, B. longum, and combinations thereof. Pseudomonas microorganism may include P. putida, P. cepacia, P. fluorescens, and combinations thereof.

In a specific embodiment, the biofertilizer composition contains (a) a mixture of live lactic acid bacteria at cumulative titer of 20,000-50,000 cfu/mL including (i) Bacillus subtilis, (ii) a Lactobacillus sp., (iii) a Bifidobacterium sp., (iv) a Lactococcus sp., and (v) Streptococcus thermophilus; (b) Saccharomyces cerevisiae NCYC Sc47; and (c) >300,000 cfu/mL each of (i) Bifidobacterium animalis DSM 16284, (ii) Lactobacillus acidophilus CECT 4529, and (iii) Bacillus subtilus ATCC PTA 6737. In a more specific embodiment, the biofertilizer composition contains (a) a mixture of live lactic acid bacteria at cumulative titer of 20,000-50,000 cfu/mL including (i) Bacillus subtilis, (ii) a Lactobacillus sp., (iii) a Bifidobacterium sp., (iv) a Lactococcus sp., and (v) Streptococcus thermophilus; (b) Saccharomyces cerevisiae NCYC Sc47; (c) >300,000 cfu/mL each of (i) Bifidobacterium animalis DSM 16284, (ii) Lactobacillus acidophilus CECT 4529, and (iii) Bacillus subtilus ATCC PTA 6737; and (d) fermented molasses.

In another specific embodiment, the biofertilizer composition contains (a) a mixture of live lactic acid bacteria at cumulative titer of 20,000-50,000 cfu/mL including (i) Bacillus subtilis, (ii) a Lactobacillus sp., (iii) a Bifidobacterium sp., (iv) a Lactococcus sp., and (v) Streptococcus thermophilus; (b) Saccharomyces cerevisiae PCM KKP 2059p; and (c) >300,000 cfu/mL each of (i) Lactococcus lactis PCM B/00039, (ii) Lactobacillus casei PCM B/00080, (iii) Lactobacillus plantarum PCM B/00081, and (iv) Carnobacterium divergens PCM KKP 2012p. In a more specific embodiment, the biofertilizer composition contains (a) a mixture of live lactic acid bacteria at cumulative titer of 20,000-50,000 cfu/mL including (i) Bacillus subtilis, (ii) a Lactobacillus sp., (iii) a Bifidobacterium sp., (iv) a Lactococcus sp., and (v) Streptococcus thermophilus; (b) Saccharomyces cerevisiae PCM KKP 2059p; (c) >300,000 cfu/mL each of (i) Lactococcus lactis PCM B/00039, (ii) Lactobacillus casei PCM B/00080, (iii) Lactobacillus plantarum PCM B/00081, and (iv) Carnobacterium divergens PCM KKP 2012p; and (d) fermented molasses.

In yet another specific embodiment, the biofertilizer composition contains (a) a mixture of live lactic acid bacteria at cumulative titer of 20,000-50,000 cfu/mL including (i) Bacillus subtilis, (ii) a Lactobacillus sp., (iii) a Bifidobacterium sp., (iv) a Lactococcus sp., and (v) Streptococcus thermophilus; (b) Saccharomyces cerevisiae IFO 0203; and (c) >300,000 cfu/mL of Lactobacillus rhamnosus ATCC 7469. In a more specific embodiment, the biofertilizer composition contains (a) a mixture of live lactic acid bacteria at cumulative titer of 20,000-50,000 cfu/mL including (i) Bacillus subtilis, (ii) a Lactobacillus sp., (iii) a Bifidobacterium sp., (iv) a Lactococcus sp., and (v) Streptococcus thermophilus; (b) Saccharomyces cerevisiae IFO 0203; (c) >300,000 cfu/mL of Lactobacillus rhamnosus ATCC 7469; and (d) fermented molasses.

In some embodiments, the microorganism consortium in culture medium contains non-pathogenic gram-positive Bacilli bacteria. In more specific embodiments, the non-pathogenic gram-positive Bacilli bacteria of the biofertilizer composition contains one or more of Bifidobacterium actinocoloniiforme, Bifidobacterium adolescentis, Bifidobacterium angulatum, Bifidobacterium animalis, Bifidobacterium aquikefiri, Bifidobacterium asteroides, Bifidobacterium biavatii, Bifidobacterium bifidum, Bifidobacterium bohemicum, Bifidobacterium bombi, Bifidobacterium boum, Bifidobacterium breve, Bifidobacterium callitrichos, Bifidobacterium catenulatum, Bifidobacterium choerinum, Bifidobacterium commune, Bifidobacterium coryneforme, Bifidobacterium cuniculi, Bifidobacterium crudilactis, Bifidobacterium denticolens, Bifidobacterium dentium, Bifidobacterium eulemuris, Bifidobacterium faecale, Bifidobacterium gallicum, Bifidobacterium gallinarum, Bifidobacterium hapali, Bifidobacterium indicum, Bifidobacterium inopinatum, Bifidobacterium kashiwanohense, Bifidobacterium infantis, Bifidobacterium lemurum, Bifidobacterium longum, Bifidobacterium magnum, Bifidobacterium merycicum, Bifidobacterium minimum, Bifidobacterium mongoliense, Bifidobacterium moukalabense, Bifidobacterium myosotis, Bifidobacterium pseudocatenulatum, Bifidobacterium pseudolongum, Bifidobacterium psychraerophilum, Bifidobacterium pullorum, Bifidobacterium reuteri, Bifidobacterium ruminantium, Bifidobacterium saguini, Bifidobacterium scardovii, Bifidobacterium stellenboschense, Bifidobacterium stercoris, Bifidobacterium saeculare, Bifidobacterium subtile, Bifidobacterium thermacidophilum, Bifidobacterium thermophilum, Bifidobacterium tissieri, and Bifidobacterium tsurumiense, Bifidobacterium animalis ssp. animalis (DSM 16284), Bifidobacterium longum subsp. Suis (DSM 20211), Bifidobacterium animalis subsp. animalis (DSM 20104), Bifidobacterium animalis subsp. lactis (DSM 20105), and Bifidobacterium animalis subsp. lactis (ATCC 27536), Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylotrophicus, Lactobacillus amylovorus, Lactobacillus animalis, Lactobacillus antri, Lactobacillus apodemi, Lactobacillus aviarius, Lactobacillus bifermentans, Lactobacillus brevis, Lactobacillus buchneri, Lactobacillus camelliae, Lactobacillus casei, Lactobacillus catenaformis, Lactobacillus ceti, Lactobacillus coleohominis, Lactobacillus collinoides, Lactobacillus composti, Lactobacillus concavus, Lactobacillus coryniformis, Lactobacillus crispatus, Lactobacillus crustorum, Lactobacillus curvatus, Lactobacillus delbrueckii subsp. bulgaricus, Lactobacillus delbrueckii subsp. delbrueckii, Lactobacillus delbrueckii subsp. lactis, Lactobacillus dextrinicus, Lactobacillus diolivorans, Lactobacillus equi, Lactobacillus equigenerosi, Lactobacillus farraginis, Lactobacillus farciminis, Lactobacillus fermentum, Lactobacillus fornicalis, Lactobacillus fructivorans, Lactobacillus frumenti, Lactobacillus fuchuensis, Lactobacillus gallinarum, Lactobacillus gasseri, Lactobacillus gastricus, Lactobacillus ghanensis, Lactobacillus graminis, Lactobacillus hammesii, Lactobacillus hamsteri, Lactobacillus harbinensis, Lactobacillus hayakitensis, Lactobacillus helveticus, Lactobacillus hilgardii, Lactobacillus homohiochii, Lactobacillus iners, Lactobacillus ingluviei, Lactobacillus intestinalis, Lactobacillus jensenii, Lactobacillus johnsonii, Lactobacillus kalixensis, Lactobacillus kefiranofaciens, Lactobacillus kefiri, Lactobacillus kimchii, Lactobacillus kitasatonis, Lactobacillus kunkeei, Lactobacillus leichmannii, Lactobacillus lindneri, Lactobacillus malefermentans, Lactobacillus mali, Lactobacillus manihotivorans, Lactobacillus mindensis, Lactobacillus mucosae, Lactobacillus murinus, Lactobacillus nagelii, Lactobacillus namurensis, Lactobacillus nantensis, Lactobacillus oligofermentans, Lactobacillus oris, Lactobacillus panis, Lactobacillus pantheris, Lactobacillus parabrevis, Lactobacillus parabuchneri, Lactobacillus paracasei, Lactobacillus paracollinoides, Lactobacillus parafarraginis, Lactobacillus parakefiri, Lactobacillus paralimentarius, Lactobacillus paraplantarum, Lactobacillus pentosus, Lactobacillus perolens, Lactobacillus plantarum, Lactobacillus ponti s, Lactobacillus protectus, Lactobacillus psittaci, Lactob acillus rennini, Lactobacillus reuteri, Lactobacillus rhamnosus, Lactobacillus rimae, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus satsumensis, Lactobacillus secaliphilus, Lactobacillus sharpeae, Lactobacillus siliginis, Lactobacillus spicheri, Lactobacillus suebicus, Lactobacillus thailandensis, Lactobacillus ultunensis, Lactobacillus vaccinostercus, Lactobacillus vaginalis, Lactobacillus versmoldensis, Lactobacillus vini, Lactobacillus vitulinus, Lactobacillus zeae, and Lactobacillus zymae, Lactobacillus casei DSM 28872, Lactobacillus casei PCM B/00080, Lactobacillus casei DSM 28872, Lactobacillus plantarum KKP/593/p, Lactobacillus plantarum KKP/788/p, Lactobacillus plantarum PCM B/00081, Lactobacillus plantarum (NCIMB 41638), Lactobacillus plantarum DSM 29024, Lactobacillus plantarum (ATCC 55943), Lactobacillus plantarum (ATCC 55944), Lactobacillus plantarum (ATCC PTSA-6139), Lactobacillus plantarum (CNCM 1-3235), Lactobacillus plantarum DSM 11672, Lactobacillus plantarum (DSM 12836), Lactobacillus plantarum (DSM 12837), Lactobacillus plantarum (DSM 16565), Lactobacillus plantarum (DSM 16568), Lactobacillus plantarum (DSM 18112), Lactobacillus plantarum (DSM 18113), Lactobacillus plantarum (DSM 18114), Lactobacillus plantarum (DSM 19457), Lactobacillus plantarum (DSM 21762), Lactobacillus plantarum (DSM 23375), Lactobacillus plantarum (DSM 29025), Lactobacillus plantarum (DSM 3676), Lactobacillus plantarum (DSM 3677), Lactobacillus plantarum (DSM 8862), Lactobacillus plantarum (DSM 8866), Lactobacillus plantarum (LMG-21295), Lactobacillus plantarum (NCIMB 30083), Lactobacillus plantarum (NCIMB 30084), Lactobacillus plantarum (NCIMB 30084), Lactobacillus plantarum (NCIMB 30084), Lactobacillus plantarum (NCIMB 30236), Lactobacillus plantarum (NCIMB 41028), Lactobacillus plantarum (NCIMB 42150), Lactobacillus plantarum (VTT E-78076), Lactobacillus plantarum C KKP/788/p, Lactobacillus plantarum CECT 4528, Lactobacillus plantarum CECT 4528, Lactobacillus plantarum K KKP/593/p, Lactobacillus plantarum LP287, Lactobacillus plantarum LP329, Lactobacillus plantarum LP329, Lactobacillus plantarum NCIMB 30238, Lactobacillus buchneri KKP/907/p, Lactobacillus buchneri (DSM 22963), Lactobacillus buchneri (DSM 12856), Lactobacillus buchneri (DSM 13573), Lactobacillus buchneri CCM 1819, Lactobacillus buchneri (DSM 16774), Lactobacillus buchneri DSM 22501, Lactobacillus buchneri LN 40177, Lactobacillus buchneri LN4637, Lactobacillus buchneri LN 40177, Lactobacillus buchneri NCIMB 40788, Lactobacillus acidophilus CECT 4529, Lactobacillus acidophilus NBIMCC 8242, Lactobacillus rhamnosus (NCIMB 41640), Lactobacillus rhamnosus (NCIMB 30121), Lactobacillus rhamnosus DSM 29226, Lactobacillus rhamnosus DSM 7133, Lactobacillus rhamnosus (CNCM-I-3698), Lactobacillus rhamnosus ATCC 7469, Lactobacillus fermentum (NCIMB 41636), Lactobacillus brevis (DSM 12835), Lactobacillus brevis (DSM 21982), Lactobacillus brevis (DSM 12835), Lactobacillus brevis DSMZ 16680, Lactobacillus paracasei (DSM 16245), Lactobacillus paracasei (DSM 16773), and Lactobacillus paracasei NCIMB 30151, Lactococcus chungangensis, Lactococcus formosensis, Lactococcus fujiensis, Lactococcus garvieae, Lactococcus hircilactis, Lactococcus lactis, Lactococcus laudensis, Lactococcus nasutitermitis, Lactococcus piscium, Lactococcus plantarum, Lactococcus raffinolactis, and Lactococcus taiwanensis, Lactococcus lactis PCM B/00039, Lactococcus lactis (DSM 11037), Lactococcus lactis (NCIMB 30117), and Lactococcus lactis (NCIMB 30160), Bacillus subtilis, Bacillus licheniformis, and Bacillus amyloliquefaciens, Bacillus subtilis (DSM 5750), Bacillus subtilis C-3102 (DSM 15544), Bacillus subtilis (ATCC PTA-6737), Bacillus subtilis (LMG 5-15136, Bacillus subtilis (DSM 28343), Bacillus subtilis (DSM 27273), Bacillus subtilis DS098, Bacillus subtilis MBS-BS-01, Bacillus subtilis (ATCC 2107), Bacillus subtilis DSM 17299, Bacillus subtilis (CBS 117162), Bacillus licheniformis (DSM 5749), Bacillus licheniformis DSM 28710, Bacillus licheniformis (DSM 19670), Bacillus licheniformis (DSM 21564), Bacillus licheniformis ATCC 53757, Bacillus amyloliquefaciens CECT 5940, Bacillus amyloliquefaciens (DSM 9553), Bacillus amyloliquefaciens (DSM 9554), Bacillus amyloliquefaciens (PTA-6507), Bacillus amyloliquefaciens (NRRL B-50013), Bacillus amyloliquefaciens (NRRL B-50104), Bacillus amyloliquefaciens SD80, and Bacillus amyloliquefaciens (ATCC 3978), Carnobacterium alterfunditum, Carnobacterium divergens, Carnobacterium funditium, Carnobacterium gallinarum, Carnobacterium iners, Carnobacterium inhibens, Carnobacterium jeotgali, Carnobacterium maltaromaticum, Carnobacterium mobile, Carnobacterium piscicola, Carnobacterium pleistocenium, and Carnobacterium viridans, Carnobacterium divergens PCM KKP 2012p, Streptococcus thermophilus, and Streptococcus thermophilus NBIMCC 8253, Streptococcus thermophilus CNRZ1066, Streptococcus thermophilus LMG13811, Enterococcus faecium, Enterococcus faecalis, Enterococcus faecium CECT 4515, Enterococcus faecium CCM 6226, Enterococcus faecium CNCM 1-3236, Enterococcus faecium DSM 22502, Enterococcus faecium NCIMB 10415, Enterococcus faecium SF202, Enterococcus faecium SF301, Enterococcus faecium DSM 7134, Enterococcus faecium DSM 10663, Enterococcus faecium NCIMB 11181, Enterococcus faecium DSM 21913, and/or Enterococcus faecium NBIMCC 8270.

In some embodiments, the microorganism consortium in culture medium contains at least one useful microorganism selected from a species of Alcaligenes sp., Aerobacter aerogenes, Achromobacter sp., Acinetobacter sp., Actinomadura oligospora, Agrobacterium sp., Azospirillum sp., Bacillus sp., Bacillus circulans, B. cereus, B. fusiformis, B. pumilis, B. megaterium, B. mycoides, B. polymyxa Paenibacillus polymyxa, B. coagulans, B. chitinolyticus Paenibacillus chitinolyticus, B. subtilis, Bacillus subtilis natto, Bradyrhizobium sp., Brevibacterium sp., Citrobacter sp., Pseudomonas sp., P putida, P. striata, P. fluorescens, P. calcis, Flavobacterium sp., Nitrosomonas sp., Erwinia sp., Micrococcus sp., Escherichia intermedia, Enterobacter asburiae, Serratia phosphoticum, Nitrobacter sp., Thiobacillus ferroxidans, T. thioxidans, Rhizobium meliloti, Xanthomonas sp., Aspergillus awamori, A. niger, A. tereus, A. flavus, A. nidulans, A. foetidus, A. wentii. Fusarium oxysporum, Alternaria teneius, Achrothcium sp. Penicillium digitatum, P lilacinium, P balaji, P. funicolosum, Cephalosporium sp. Cladosprium sp., Curvularia lunata, Cunnighamella, Candida sp., Chaetomium globosum, Humicola inslens, Humicola lanuginosa, Helminthosporium sp., Paecilomyces fusisporous, Pythium sp., Phoma sp., Populospora mytilina, Myrothecium roridum, Morteirella sp., Micromonospora sp., Oideodendron sp., Rhizoctonia solani, Rhizopus sp., Mucor sp., Trichoderma viridae, Torula thermophila, Schwanniomyces occidentalis, Sclerotium rolfsii, Actinomyces, Streptomyces., Anabena sp., Calothrix braunii, Nostoc sp., Scytonema sp., or Glomus fasciculatum.

In some embodiments, the microorganism consortium in culture medium may optionally contain at least one useful microorganism selected from Bifidobacterium bifidum Tissier Orla-Jensen ATCC 29521, Bifidobacterium bifidum Tissier Orla-Jensen ATCC BAA-2850, Bifidobacterium bifidum Tissier Orla-Jensen ATCC 11863, Bifidobacterium bifidum Tissier Orla-Jensen ATCC 35914, Bifidobacterium bifidum Tissier Orla-Jensen ATCC 15696, or Bifidobacterium animalis subsp. lactis ATCC 700541.

As well, the present microorganism consortium in culture medium may also or instead optionally contain at least one useful microorganism selected from Bifidobacterium longum subsp. infantis Reuter Mattarelli et al. ATCC 15697, Bifidobacterium longum subsp. longum Reuter ATCC 15707, Bifidobacterium longum subsp. suis Matteuzzi et al. Mattarelli et al. ATCC 27533, Bifidobacterium longum subsp. infantis Reuter Mattarelli et al. ATCC 25962, Bifidobacterium longum subsp. infantis Reuter Mattarelli et al. ATCC 15702, Bifidobacterium longum Reuter ATCC BAA-999, Bifidobacterium longum subsp. suis Matteuzzi et al. Mattarelli et al. ATCC 27532, Bifidobacterium longum Reuter ATCC 15708, Bifidobacterium longum Reuter ATCC 55815, Bifidobacterium longum Reuter ATCC 55813, Bifidobacterium longum Reuter ATCC 55818, Bifidobacterium longum Reuter ATCC 55817, Bifidobacterium longum subsp. suis (Matteuzzi et al.) Mattarelli et al. ATCC 27531, Bifidobacterium longum Reuter ATCC 51870, Bifidobacterium longum Reuter ATCC 35183, Bifidobacterium longum subsp. infantis Reuter Mattarelli et al. ATCC 15697D-5, Bifidobacterium animalis subsp. animalis Mitsuoka Scardovi and Trovatelli ATCC 25527, Bifidobacterium longum subsp. infantis Reuter Mattarelli et al. ATCC 17930, Bifidobacterium longum subsp. longum Reuter ATCC BAA-2753, Bifidobacterium longum Reuter ATCC 55814, or Bifidobacterium longum Reuter ATCC 55816.

Moreover, in some embodiments, the microorganism consortium in culture medium may include one or more useful microorganisms selected from Lactobacillus acidophilus Moro Hansen and Mocquot ATCC 4356, Lactobacillus acidophilus Moro Hansen and Mocquot ATCC 4355, Lactobacillus acidophilus Moro Hansen and Mocquot ATCC 4796, Lactobacillus acidophilus Moro Hansen and Mocquot ATCC 4357, Lactobacillus acidophilus Moro Hansen and Mocquot ATCC BAA-2832, Lactobacillus acidophilus Moro Hansen and Mocquot ATCC 53546, Lactobacillus acidophilus Moro Hansen and Mocquot ATCC 53544, Lactobacillus acidophilus Moro Hansen and Mocquot ATCC 11975, Lactobacillus acidophilus Moro Hansen and Mocquot ATCC 314, Lactobacillus acidophilus Moro Hansen and Mocquot ATCC 832, Lactobacillus acidophilus Moro Hansen and Mocquot ATCC 9224, Lactobacillus acidophilus Moro Hansen and Mocquot ATCC 43121, Lactobacillus acidophilus Moro Hansen and Mocquot ATCC 53671, Lactobacillus acidophilus: B6T7 PTA-4482, Lactobacillus acidophilus Moro Hansen and Mocquot ATCC BAA-2845, Lactobacillus acidophilus Moro Hansen and Mocquot ATCC 4357D-5, Lactobacillus animalis LA51 Deposited as Lactobacillus acidophilus PTA-6750, Lactobacillus acidophilus isolated from bovine feed LA45 PTA-6749, Lactobacillus acidophilus M35 PTA-6751, Lactobacillus acidophilus L411 PTA-6820, Lactobacillus gallinarum Fujisawa et al. ATCC 33199, Lactobacillus amylovorus Nakamura ATCC 33198, Lactobacillus gasseri Lauer and Kandler ATCC 19992, Lactobacillus johnsonii Fujisawa et al. ATCC 11506, Lactobacillus johnsonii Fujisawa et al. ATCC 33200, Lactobacillus johnsonii Fujisawa et al. ATCC 332, Lactobacillus rhamnosus Hansen Collins et al. ATCC 53103, Lactobacillus crispatus Brygoo and Aladame Moore and Holdeman ATCC 33197, Lactobacillus crispatus Brygoo and Aladame Moore and Holdeman ATCC 53545, Lactobacillus rhamnosus Hansen Collins et al. ATCC 21052, Bacillus sp. ATCC 31283 (Lactobacillus acidophilus Moro Hansen and Mocquot), Lactobacillus casei Orla-Jensen Hansen and Lessel ATCC 4646 (Lactobacillus acidophilus Moro Hansen and Mocquot), Lactobacillus gasseri Lauer and Kandler ATCC 9857 (Lactobacillus acidophilus Moro Hansen and Mocquot), Lactobacillus fermentum Beijerinck ATCC 11976 (Lactobacillus acidophilus Moro Hansen and Mocquot), Lactobacillus gasseri Lauer and Kandler ATCC 29601 (Lactobacillus acidophilus Moro Hansen and Mocquot), Lactobacillus paracasei subsp. paracasei Collins et al. ATCC 11974 (Lactobacillus acidophilus Moro Hansen and Mocquot), Lactobacillus johnsonii Fujisawa et al. ATCC 53672 (Lactobacillus acidophilus Moro Hansen and Mocquot), or Lactobacillus crispatus Brygoo and Aladame Moore and Holdeman ATCC 55221 (Lactobacillus acidophilus Moro Hansen and Mocquot).

In some embodiments, the microorganism consortium in culture medium of the present disclosure may optionally comprise one or more useful strains of Lactobacillus bulgaricus (Lactobacillus delbrueckii subsp. bulgaricus) selected from Lactobacillus delbrueckii subsp. bulgaricus Orla-Jensen Weiss et al. ATCC 11842 (Lactobacillus bulgaricus Orla-Jensen Rogosa and Jensen), Lactobacillus bulgaricus Orla-Jensen Rogosa and Hansen ATCC BAA-2844, Lactobacillus delbrueckii subsp. bulgaricus Orla-Jensen Weiss et al. ATCC BAA-365, Lactobacillus delbrueckii subsp. bulgaricus Orla-Jensen Weiss et al. ATCC 11842D-5, Lactobacillus delbrueckii subsp. bulgaricus Orla-Jensen Weiss et al. ATCC BAA-365D, Lactobacillus helveticus Orla-Jensen Bergey et al. ATCC 521, Lactobacillus helveticus Orla-Jensen Bergey et al. ATCC 27558, Lactobacillus helveticus Orla-Jensen Bergey et al. ATCC 11977, Lactobacillus helveticus Orla-Jensen Bergey et al. ATCC 7995, Lactobacillus acidophilus Moro Hansen and Mocquot ATCC 9224, Lactobacillus sp. ATCC 7517, Lactobacillus sp. ATCC 12278, Lactobacillus sp. ATCC 8001, Lactobacillus leichmannii Henneberg Bergey et al. ATCC 21815, Lactobacillus helveticus Orla-Jensen Bergey et al. ATCC 33409, Lactobacillus helveticus Orla-Jensen Bergey et al. ATCC 55163, Lactobacillus sp. ATCC 13866, Lactobacillus delbrueckii subsp. lactis Orla-Jensen Weiss et al. ATCC 10705, or Lactobacillus helveticus Orla-Jensen Bergey et al. ATCC 7994.

Additionally, in some embodiments, the microorganism consortium in culture medium may comprise one or more useful microorganisms selected from Lactobacillus casei DSM 28872, Lactobacillus casei PCM B/00080, or Lactobacillus casei DSM 28872.

In some embodiments, the microorganism consortium in culture medium may comprise one or more useful microorganisms selected from Lactobacillus fermentum Beijerinck ATCC 14932, Lactobacillus fermentum Beijerinck ATCC 14931, Lactobacillus fermentum Beijerinck ATCC 11739, Lactobacillus fermentum Beijerinck ATCC 9338, Lactobacillus fermentum Beijerinck ATCC 11976, Lactobacillus fermentum Beijerinck ATCC 23271, Lactobacillus fermentum Beijerinck ATCC 11740, Lactobacillus fermentum Beijerinck ATCC 8289, Lactobacillus fermentum B-54 55884, Lactobacillus reuteri Kandler et al. ATCC 23272 (Lactobacillus fermentum Beijerinck), or Lactobacillus fermentum Beijerinck ATCC BAA-2842.

Moreover, in some embodiments, the microorganism consortium in culture medium may comprise one or more useful microorganisms selected from Lactobacillus lactis: DJ6 PTA-6102, Lactobacillus lactis: CI15 PTA-6101, Lactococcus lactis subsp. lactis Lister Schleifer et al. ATCC 15577, Lactobacillus delbrueckii subsp. lactis Orla-Jensen Weiss et al. ATCC 8000D, Lactobacillus delbrueckii subsp. lactis Orla-Jensen Weiss et al. ATCC 12315, Lactobacillus delbrueckii subsp. lactis Orla-Jensen Weiss et al. ATCC 12314, Lactobacillus delbrueckii subsp. lactis Orla-Jensen Weiss et al. ATCC 10705, Lactobacillus delbrueckii subsp. lactis Orla-Jensen Weiss et al. ATCC 11061, Lactobacillus delbrueckii subsp. lactis Orla-Jensen Weiss et al. ATCC 21051, Lactobacillus delbrueckii subsp. lactis Orla-Jensen Weiss et al. ATCC 11983, Lactobacillus delbrueckii subsp. lactis Orla-Jensen Weiss et al. ATCC 8000, Lactobacillus delbrueckii subsp. lactis Orla-Jensen Weiss et al. ATCC 15808, Lactobacillus leichmannii Henneberg Bergey et al. ATCC 10697, Lactobacillus leichmannii Henneberg Bergey et al. ATCC 39538, Leuconostoc lactis Garvie ATCC 15520, Lactobacillus leichmannii Henneberg Bergey et al. ATCC 7830, Lactobacillus leichmannii Henneberg Bergey et al. ATCC 7830, Lactobacillus leichmannii Henneberg Bergey et al. ATCC 7830D-5, Lactobacillus helveticus Orla-Jensen Bergey et al. ATCC 27558, Lactobacillus helveticus Orla-Jensen Bergey et al. ATCC 11977, Lactobacillus helveticus Orla-Jensen Bergey et al. ATCC 55163, Lactobacillus leichmannii Henneberg Bergey et al. ATCC 21815, Lactobacillus helveticus Orla-Jensen Bergey et al. ATCC 7994, or Lactobacillus helveticus Orla-Jensen Bergey et al. ATCC 33409.

As well, in some embodiments, the present microorganism consortium in culture medium may comprise one or more useful microorganisms selected from Lactobacillus plantarum KKP/593/p, Lactobacillus plantarum KKP/788/p, Lactobacillus plantarum PCM B/00081, Lactobacillus plantarum (NCIMB 41638), Lactobacillus plantarum DSM 29024, Lactobacillus plantarum (ATCC 55943), Lactobacillus plantarum (ATCC 55944), Lactobacillus plantarum (ATCC PTSA-6139), Lactobacillus plantarum (CNCM 1-3235), Lactobacillus plantarum DSM 11672, Lactobacillus plantarum (DSM 12836), Lactobacillus plantarum (DSM 12837), Lactobacillus plantarum (DSM 16565), Lactobacillus plantarum (DSM 16568), Lactobacillus plantarum (DSM 18112), Lactobacillus plantarum (DSM 18113), Lactobacillus plantarum (DSM 18114), Lactobacillus plantarum (DSM 19457), Lactobacillus plantarum (DSM 21762), Lactobacillus plantarum (DSM 23375), Lactobacillus plantarum (DSM 29025), Lactobacillus plantarum (DSM 3676), Lactobacillus plantarum (DSM 3677), Lactobacillus plantarum (DSM 8862), Lactobacillus plantarum (DSM 8866), Lactobacillus plantarum (LMG-21295), Lactobacillus plantarum (NCIMB 30083), Lactobacillus plantarum (NCIMB 30084), Lactobacillus plantarum (NCIMB 30084), Lactobacillus plantarum (NCIMB 30084), Lactobacillus plantarum (NCIMB 30236), Lactobacillus plantarum (NCIMB 41028), Lactobacillus plantarum (NCIMB 42150), Lactobacillus plantarum (VTT E-78076), Lactobacillus plantarum C KKP/788/p, Lactobacillus plantarum CECT 4528, Lactobacillus plantarum CECT 4528, Lactobacillus plantarum K KKP/593/p, Lactobacillus plantarum LP287, Lactobacillus plantarum LP329, Lactobacillus plantarum LP329, and Lactobacillus plantarum NCIMB 30238.

Additionally, in some embodiments, the microorganism consortium in culture medium may comprise one or more useful microorganisms selected from Lactobacillus parafarraginis NCIMB 15108 or Lactobacillus parafarraginis NCIMB 702943.

As well, in some embodiments, the microorganism consortium in culture medium may comprise one or more useful microorganisms selected from Lactobacillus rapi DSM-19907, Lactobacillus rapi Type Strain JCM 15042 NRIC 0743, Leuconostoc rapi DSM-27776, or Leuconostoc rapi Type Strain LMG 27676.

Moreover, in some embodiments, the present microorganism consortium in culture medium may comprise one or more useful strains of Acetobacter ghanensis, including but not limited to Acetobacter ghanensis DSM-18895.

In some embodiments, the microorganism consortium in culture medium may optionally comprise one or more useful microorganisms selected from the group comprising Gluconacetobacter diazotrophicus Gillis et al. Yamada et al. ATCC 49037, Gluconacetobacter diazotrophicus Gillis et al. Yamada et al. ATCC 49039, Gluconacetobacter diazotrophicus Gillis et al. Yamada et al. ATCC 49038, or Gluconacetobacter diazotrophicus Gillis et al. Yamada et al. ATCC 49037D-5.

In some embodiments, the microorganism consortium in culture medium may optionally contain at least one useful microorganism selected from the group comprising Alcaligenes faecalis ATCC 8750, Genomic DNA from Alcaligenes faecalis subsp. faecalis strain 16 ATCC 8750D-5, Alcaligenes faecalis subsp. faecalis Castellani and Chalmers ATCC 35655, Alcaligenes faecalis Castellani and Chalmers ATCC 53602, Alcaligenes faecalis subsp. faecalis Castellani and Chalmers ATCC 19018, Alcaligenes faecalis subsp. faecalis Castellani and Chalmers ATC 35655, Alcaligenes faecalis subsp. faecalis Castellani and Chalmers ATCC 27260, Alcaligenes faecalis subsp. faecalis Castellani and Chalmers ATCC 49677, Alcaligenes faecalis subsp. faecalis Castellani and Chalmers ATCC 33950, Alcaligenes faecalis subsp. faecalis Castellani and Chalmers ATCC 15554, Alcaligenes faecalis subsp. faecalis Castellani and Chalmers ATCC 43161, Alcaligenes faecalis Castellani and Chalmers ATCC 15101, Alcaligenes faecalis subsp. faecalis Castellani and Chalmers ATCC 8748, Alcaligenes faecalis subsp. faecalis Castellani and Chalmers ATCC 15246, Alcaligenes faecalis subsp. faecalis Castellani and Chalmers ATCC 19209, Alcaligenes faecalis Castellani and Chalmers ATCC 700596, or Alcaligenes faecalis subsp. faecalis Castellani and Chalmers ATCC 33585.

In some embodiments, the microorganism consortium in culture medium may optionally contain at least one useful microorganism selected from the group comprising Achromobacter xylosoxidans ex Yabuuchi and Ohyama Yabuuchi and Yano ATCC 27061, Achromobacter xylosoxidans ex Yabuuchi and Ohyama Yabuuchi and Yano ATCC 212, Achromobacter xylosoxidans ex Yabuuchi and Ohyama Yabuuchi and Yano ATCC 27062, Achromobacter xylosoxidans ex Yabuuchi and Ohyama Yabuuchi and Yano ATCC 27063, Achromobacter xylosoxidans ex Yabuuchi and Ohyama Yabuuchi and Yano ATCC 31040, Achromobacter xylosoxidans ex Yabuuchi and Ohyama Yabuuchi and Yano ATCC 9220, Achromobacter xylosoxidans ex Yabuuchi and Ohyama Yabuuchi and Yano ATCC 15446, Achromobacter denitrificans Ruger and Tan Coenye et al. ATCC 15173, Achromobacter denitrificans Ruger and Tan Coenye et al. ATCC 55564, Achromobacter denitrificans Ruger and Tan Coenye et al. ATC 13138, Achromobacter piechaudii Kiredjian et al. Yabuuchi et al. ATC 43552, or Achromobacter ruhlandii Packer and Vishniac Yabuuchi et al. ATCC 15749.

Additionally, in some embodiments, the microorganism consortium in culture medium may optionally contain at least one useful microorganism selected from the group comprising Acinetobacter lwoffii Audureau Brisou ATCC 15309, Acinetobacter lwoffii Audureau Brisou ATCC 17925, Acinetobacter sp. ATCC 39769, Acinetobacter sp. ATCC 39770, Acinetobacter baumannii Bouvet and Grimont ATCC 19606, Acinetobacter baumannii Bouvet and Grimont ATCC 19606, Acinetobacter calcoaceticus Beijerinck Baumann et al. ATCC 23055, Acinetobacter sp. genomospecies 3 ATCC 19004, Acinetobacter junii Bouvet and Grimont ATCC 17908, Acinetobacter guillouiae Nemec et al. ATCC 11171, Acinetobacter bereziniae Nemec et al. ATCC 17924, Acinetobacter haemolyticus Bouvet and Grimont ATCC 17906, Acinetobacter johnsonii Bouvet and Grimont ATCC 17909, Acinetobacter sp. genomospecies 9 ATCC 17910, Acinetobacter sp. genomospecies 9 ATCC 17984, Acinetobacter sp. genomospecies 9 ATCC 17968, Acinetobacter sp. genomospecies 3 ATCC 17922, Acinetobacter sp. genomospecies 6 ATCC 17979, or Acinetobacter sp. genomospecies 9 ATCC 9957, Acinetobacter calcoaceticus Beijerinck Baumann et al. ATCC 23055, Acinetobacter calcoaceticus Beijerinck Baumann et al. ATCC 14987, Acinetobacter calcoaceticus Beijerinck Baumann et al. ATCC 17902, Acinetobacter calcoaceticus Beijerinck Baumann et al. ATCC 31926, Acinetobacter calcoaceticus Beijerinck Baumann et al. ATCC BAA-347, Acinetobacter baumannii Bouvet and Grimont ATCC 49466, Acinetobacter baylyi ATCC 33305, Acinetobacter baumannii ATCC 43498, pSC161RM O/P17 ATCC 40897, Acinetobacter sp. ATCC 39647, Acinetobacter baylyi ATCC 33304, Acinetobacter sp. ATCC 43608, Acinetobacter sp. ATCC 39648, Acinetobacter sp. ATCC 49823, Acinetobacter calcoaceticus Beijerinck Baumann et al. ATCC 19638, Acinetobacter baumannii Bouvet and Grimont ATCC 51432, Acinetobacter sp. ATCC 33969, Acinetobacter sp. ATCC 49467, Acinetobacter sp. ATCC 49468, Acinetobacter sp. ATC 31299, Acinetobacter calcoaceticus Beijerinck Baumann et al. ATCC BAA-346, or Acinetobacter sp. ATCC 33951.

In some embodiments, the microorganism consortium in culture medium may optionally contain at least one useful microorganism selected from the group comprising Azospirillum brasilense Tarrand et al. ATCC 35213, Azospirillum brasilense Tarrand et al. ATCC 29729, Azospirillum brasilense Tarrand et al. ATCC 29710, Azospirillum brasilense Tarrand et al. ATCC 29711, Azospirillum brasilense Tarrand et al. ATCC 29145, Azospirillum brasilense Tarrand et al. ATCC 35212, Azospirillum lipoferum Beijerinck Tarrand et al. ATCC 29707, Azospirillum lipoferum Beijerinck Tarrand et al. ATCC 29709, Azospirillum lipoferum Beijerinck Tarrand et al. ATCC 29731, Azospirillum lipoferum Beijerinck Tarrand et al. ATCC 29708, or Azospirillum irakense Khammas et al. ATCC 51182.

In one or more embodiments, the at least one useful microorganism may be Actinomadura sp. is Actinomadura oligospora Mertz and Yao ATCC 43269, or Bacillus circulans Jordan ATCC 19439,

Moreover, in some embodiments, the microorganism consortium in culture medium may optionally contain at least one useful microorganism selected from the group comprising Bacillus cereus Frankland and Frankland ATCC 14579, Bacillus cereus Frankland and Frankland ATCC 10876, Bacillus cereus Frankland and Frankland ATCC 13061, Bacillus cereus Frankland and Frankland ATCC 10876, Bacillus cereus Frankland and Frankland ATCC 11778, Bacillus cereus Frankland and Frankland ATCC BAA-1005, Bacillus cereus Frankland and Frankland ATCC 15816, Bacillus cereus Frankland and Frankland ATCC 15817, Bacillus cereus Frankland and Frankland ATCC BAA-512, Bacillus cereus Frankland and Frankland ATCC 23261, Bacillus cereus Frankland and Frankland ATCC 21772, Bacillus cereus Frankland and Frankland ATCC 21634, Bacillus cereus Frankland and Frankland ATCC 7004, Bacillus cereus Frankland and Frankland ATCC 19637, Bacillus cereus Frankland and Frankland ATCC 21182, Bacillus cereus Frankland and Frankland ATCC 7064, Bacillus cereus Frankland and Frankland ATCC 23260, Bacillus cereus Frankland and Frankland ATCC 4342, Bacillus cereus Frankland and Frankland ATCC 49063, Bacillus cereus Frankland and Frankland ATCC 31430, Bacillus cereus Frankland and Frankland ATCC 43881, Bacillus cereus Frankland and Frankland ATCC 49064, Bacillus cereus Frankland and Frankland ATCC 14603, Bacillus cereus Frankland and Frankland ATCC 55000, Bacillus cereus Frankland and Frankland ATCC 9592, Bacillus cereus Frankland and Frankland ATCC 12480, Bacillus cereus Frankland and Frankland ATCC 21366, Bacillus cereus Frankland and Frankland ATCC 10702, Bacillus cereus Frankland and Frankland ATCC 700282, Bacillus cereus Frankland and Frankland ATCC 33018, Bacillus cereus Frankland and Frankland ATCC 10987, Bacillus cereus Frankland and Frankland ATCC 53522, Bacillus cereus Frankland and Frankland ATCC 246, Bacillus cereus Frankland and Frankland ATCC 21769, Bacillus cereus Frankland and Frankland ATCC 9139, Bacillus cereus Frankland and Frankland ATCC 27522, Bacillus cereus Frankland and Frankland ATCC 33019, Bacillus cereus Frankland and Frankland ATCC 7039, Bacillus cereus Frankland and Frankland ATCC 55055, Bacillus cereus Frankland and Frankland ATCC 21768, Bacillus cereus Frankland and Frankland ATCC 27877, Bacillus cereus Frankland and Frankland ATCC 14737, Bacillus cereus Frankland and Frankland ATCC 55609, Bacillus cereus Frankland and Frankland ATCC 27348, Bacillus cereus Frankland and Frankland ATCC 9818, Bacillus cereus Frankland and Frankland ATCC 6464, Bacillus cereus Frankland and Frankland ATCC 25621, Bacillus cereus Frankland and Frankland ATCC 11950, Bacillus cereus Frankland and Frankland ATCC 14893, Bacillus cereus Frankland and Frankland ATCC 13472, Bacillus cereus Frankland and Frankland ATCC 13824, Bacillus cereus Frankland and Frankland ATCC 14579D-5, Bacillus cereus Frankland and Frankland ATCC 10987D-5, Bacillus cereus Frankland and Frankland ATCC 10702D-5, Bacillus cereus Frankland and Frankland ATCC 13061D-5, Bacillus cereus Frankland and Frankland ATCC 10876D-5, Bacteriophage of Bacillus cereus-anthracia, NikoA PTA-4171, Bacteriophage of Bacillus cereus-anthracis, DDBa PTA-4172, Bacteriophage of Bacillus cereus-anthracis, MHWa PTA-4173, or Bacillus cereus 21771.

In some embodiments, one or more useful microorganism may be selected from the group comprising Bacillus thuringiensis Berliner ATCC 10792 Strain Designations: [CCUG 7429, CIP 53.137, DSM 2046, HAMBI 478, LMG 7138, NCAIM B.01292, NCCB 70008, NRRL HD-735, VKM B-1544], Bacillus thuringiensis Berliner ATCC 19265, Bacillus thuringiensis Berliner ATCC 13367, Bacillus cereus Frankland and Frankland ATCC 21928, Bacillus thuringiensis Berliner ATCC 39152, or Bacillus thuringiensis Berliner ATCC 13366.

In some embodiments, at least one useful microorganism may be selected from a group comprising Bacillus weihenstephanensis Lechner et al. ATCC 12826, Bacillus weihenstephanensis bacteriophage ATCC 12826-B2, 31293, 31429, 31292, or Bacillus sp. ATCC 13062, P7 ATCC 75237.

In some embodiments, one or more additional microorganisms may either be present in the present composition's fermentation medium or may be added after fermentation has completed. One such microorganism may be, but is not limited to, Brevundimonas diminuta Leifson and Hugh Segers et al. ATCC 19146 or Brevundimonas diminuta Leifson and Hugh Segers et al. ATCC 19146.

In some embodiments, the microorganism consortium in culture medium may optionally contain at least one useful microorganism selected from the group comprising Bacillus licheniformis Weigmann Chester ATCC 25972 or Bacillus thuringiensis Berliner ATCC 700872.

As well, in some embodiments, the microorganism consortium in culture medium may optionally contain at least one useful microorganism selected from the group comprising Bacillus pumilus Meyer and Gottheil ATCC 7061, Bacillus pumilus Meyer and Gottheil ATCC BAA-1434, Bacillus pumilus Meyer and Gottheil ATCC 700814, Bacillus pumilus Meyer and Gottheil ATCC 19646, Bacillus pumilus Meyer and Gottheil ATCC 14884, Bacillus pumilus Meyer and Gottheil ATCC 70, Bacillus pumilus Meyer and Gottheil ATCC 31650, Bacillus pumilus Meyer and Gottheil ATCC 98, Bacillus pumilus Meyer and Gottheil ATCC 71, Bacillus pumilus Meyer and Gottheil ATCC 19164, Bacillus pumilus Meyer and Gottheil ATCC 31095, Bacillus pumilus Meyer and Gottheil ATCC 4522, Bacillus pumilus Meyer and Gottheil ATCC 945, Bacillus pumilus Meyer and Gottheil ATCC 31093, Bacillus pumilus Meyer and Gottheil ATCC 21143, Bacillus pumilus Meyer and Gottheil ATCC 27142, Bacillus pumilus Meyer and Gottheil ATCC 6632, Bacillus pumilus Meyer and Gottheil ATCC 19878, Bacillus pumilus Meyer and Gottheil ATCC 31340, Bacillus pumilus Meyer and Gottheil ATCC 19546, Bacillus pumilus Meyer and Gottheil ATCC 7065, Bacillus pumilus Meyer and Gottheil ATCC 53206, Bacillus pumilus Meyer and Gottheil ATCC 4510, Bacillus pumilus Meyer and Gottheil ATCC 19548, Bacillus pumilus Meyer and Gottheil ATCC BAA-1434, Bacillus pumilus Meyer and Gottheil ATCC 31132, Bacillus pumilus Meyer and Gottheil ATCC 15716, Bacillus pumilus Meyer and Gottheil ATCC 19646, Bacillus pumilus Meyer and Gottheil ATCC 18, Bacillus pumilus Meyer and Gottheil ATCC 4520, Bacillus pumilus Meyer and Gottheil ATCC 6631, Bacillus pumilus Meyer and Gottheil ATCC 31177, Bacillus pumilus Meyer and Gottheil ATCC 1 Strain Designations: AMC [NRS 309], Bacillus pumilus 19547, Bacillus subtilis Ehrenberg Cohn ATCC 15477 Strain Designations: M-24-1, Bacillus subtilis Ehrenberg Cohn ATCC 31098, Bacillus subtilis Ehrenberg Cohn ATCC 21951, Bacillus subtilis Ehrenberg Cohn ATCC 21008, Bacillus pumilus Meyer and Gottheil ATCC 19182, Bacillus pumilus Meyer and Gottheil ATCC 72, Bacillus subtilis 21007, Bacillus subtilis 21358, Bacillus subtilis 21005, Bacillus subtilis 21006, Bacillus subtilis 21356, Bacillus subtilis 19220, 53683, Bacillus subtilis 19219, B. subtilis 53685, Bacillus subtilis 21357, B. subtilis 21398, B. subtilis 53689, Bacillus subtilis Ehrenberg Cohn ATCC 21770, or Bacillus subtilis 19217.

Additionally, in some embodiments, the microorganism consortium in culture medium may optionally contain at least one useful microorganism selected from the group comprising Bacillus mycoides Flugge ATCC 6462 Strain Designations: NRS 273 [155, CCUG 26678, CIP 103472, DSM 2048, HAMBI 1827, LMG 7128, NCTC 12974, NRRL B-14799, NRRL B-14811], Bacillus mycoides bacteriophage ATCC 11986-B1, Bacillus mycoides Flugge ATCC 31101, Bacillus mycoides Flugge ATCC 31103, Bacillus mycoides Flugge ATCC 10206, Bacillus mycoides Flugge ATCC 21929, Bacillus mycoides Flugge ATCC 11986, Bacillus mycoides Flugge ATCC 19647, Bacillus mycoides Flugge ATCC 31102, Rhodococcus rhodochrous Zopf Tsukamura emend. Rainey et al. ATCC 27, Bacillus cereus Frankland and Frankland ATCC 11778, Rhodococcus rhodochrous Zopf Tsukamura emend. Rainey et al. ATCC 999, Bacillus mycoides Flugge ATCC 6463, Bacillus mycoides Flugge ATCC 23258, Rhodococcus rhodochrous Zopf Tsukamura emend. Rainey et al. ATCC 4004, Bacillus mycoides Flugge ATCC 21929, or Bacillus pumilus Meyer and Gottheil ATCC 19646.

In some embodiments, the microorganism consortium in culture medium may optionally contain at least one useful microorganism selected from the group comprising Bacillus coagulans Hammer ATCC 7050 Strain Designations: NRS 609 [NCIB 9365, NCTC 10334], Bacillus coagulans Hammer ATCC 12245 Strain Designations: [NCA 308], Bacillus coagulans Hammer ATCC 31284, Bacillus coagulans Hammer ATCC 53595, Bacillus coagulans Hammer ATCC 8038 Strain Designations: NCA 43P [NCIB 8080, NRS 770], Bacillus coagulans Hammer ATCC 15949 Strain Designations: NCA 4259, Bacillus coagulans Hammer ATCC 23498 Strain Designations: M-39, Bacillus coagulans Hammer ATCC 11369, Bacillus coagulans Hammer ATCC BAA-738, Bacillus coagulans Hammer ATCC 11014 Strain Designations: NRS T27 [78G], Bacillus coagulans Hammer ATCC 10545 Strain Designations: NRS 784 [NCIB 8041], pCR46 [Bacillus subtilis IS75] ATCC 67736, Bacillus cereus Frankland and Frankland ATCC 21366, Bacillus smithii Nakamura et al. ATCC 35670 Strain Designations: NRS 22 [815], Bacillus smithii Nakamura et al. ATCC 51232 Strain Designations: FRR B666 [SLS 37], or Bacillus cereus Frankland and Frankland ATCC 55055 Strain Designations: NEB 566.

Moreover, in some embodiments, the microorganism consortium in culture medium may optionally contain at least one useful microorganism selected from the group comprising Bacillus megaterium de Bary ATCC 14581 Strain Designations: [BCRC 10608, CCM 2007, CCUG 1817, CIP 66.20, DSM 32, HAMBI 2018, IAM 13418, JCM 2506, KCTC 3007, LMG 7127, NBRC 15308, NCCB 75016, NCIMB 9376, NCTC 10342, NRIC 1710, NRRL B-14308, VKM B-512], Bacillus megaterium de Bary ATCC 14581, Bacillus megaterium de Bary ATCC 13632, Bacillus megaterium de Bary ATCC 15117, Bacillus megaterium de Bary ATCC 7703, Bacillus megaterium de Bary ATCC 19135, Bacillus megaterium de Bary ATCC 33169, Bacillus megaterium de Bary pathovar cerealis ATCC 35075, Bacillus megaterium de Bary ATCC 25848, Bacillus megaterium de Bary ATCC 19213, Bacillus megaterium de Bary ATCC 6459, Bacillus megaterium de Bary ATCC 33166, Bacillus megaterium de Bary ATCC 21209, Bacillus megaterium de Bary ATCC 19160, Bacillus megaterium de Bary ATCC 33168, Bacillus megaterium de Bary ATCC 39118, Bacillus megaterium de Bary pathovar cerealis ATCC 35076, Bacillus megaterium de Bary ATCC 31294, Bacillus megaterium de Bary ATCC 13639, Bacillus megaterium de Bary ATCC 11478, Bacillus megaterium de Bary ATCC 33165, Bacillus megaterium de Bary ATCC 33729, Bacillus megaterium bacteriophage G ATCC 43725-B1, Bacillus megaterium de Bary ATCC 15451, Bacillus megaterium de Bary ATCC 7051, Bacillus megaterium de Bary ATCC 89, Bacillus megaterium de Bary ATCC 15374, Bacillus megaterium de Bary ATCC 33164, Bacillus megaterium de Bary ATCC 15127, Bacillus megaterium de Bary ATCC 15118, Bacillus megaterium de Bary ATCC 6458, Bacillus megaterium de Bary ATCC 12872, Bacillus megaterium de Bary ATCC 7052, Bacillus megaterium de Bary ATCC 21916, Bacillus megaterium de Bary ATCC 9885, Bacillus megaterium de Bary ATCC 33167, Bacillus megaterium de Bary ATCC 11562, Bacillus megaterium de Bary ATCC 25300, Bacillus megaterium de Bary ATCC 10778, Bacillus megaterium de Bary ATCC 13402, Bacillus megaterium de Bary ATCC 25833, Bacillus megaterium de Bary ATCC 21181, Bacillus megaterium de Bary ATCC 7056, Bacillus megaterium de Bary ATCC 4531, Bacillus megaterium de Bary ATCC 43725, Bacillus megaterium de Bary ATCC 27327, Bacillus megaterium de Bary ATCC 15781, Bacillus megaterium de Bary ATCC 49099, Bacillus megaterium de Bary ATCC 11561b, Bacillus megaterium de Bary ATCC 11561d, Bacillus megaterium de Bary ATCC 15450, Bacillus megaterium de Bary ATCC 49096, Bacillus megaterium de Bary ATCC 35985, Bacillus megaterium de Bary ATCC 14945, Bacillus megaterium de Bary ATCC 11561a, Bacillus megaterium de Bary ATCC 11561e, Bacillus megaterium de Bary ATCC 11561c, Bacillus megaterium de Bary ATCC 14946, Bacillus megaterium de Bary ATCC 11561, Bacillus megaterium 19161, Bacillus megaterium 15047, Bacillus megaterium 15046, Bacillus megaterium 19137, Bacillus megaterium 19218, Bacillus megaterium, SB 3112 PTA-3142, PTA-3142, Bacillus megaterium 15128, Bacillus megaterium 15177, Bacillus simplex Priest et al. ATCC 49097 Strain Designations: NRS 960 [DSM 1321], Bacillus flexus Priest et al. ATCC 49095 Strain Designations: NRS 665 [DSM 1320], Bacillus cereus Frankland and Frankland ATCC 55000 Strain Designations: B 153-2-2, Bacillus circulans Jordan ATCC 21737 Strain Designations: T5 [FERM-P 935], Bacillus simplex Priest et al. ATCC 13368 Strain Designations: 41, Bacillus sp. ATCC 13062 Strain Designations: MB-1073 [14-B22], Bacillus simplex Priest et al. ATCC 8011 Strain Designations: NRS 335 [NCTC 2597], Bacillus simplex Priest et al. ATCC 49098 Strain Designations: NRS 986 [DSM 1323], ATCC 19136, ATCC 39383, ATCC 39383, ATCC 21738, ATCC 21603, ATCC 19380, pBC16 ATCC 37338, ATCC 8245, or ATCC 51946.

In one or more embodiments, the at least one microorganism may comprise Bacillus subtilis Ehrenberg Cohn ATCC 15245 Strain Designations: 3349 [IAM 1-3].

Additionally, in some embodiments, the microorganism consortium in culture medium may optionally contain at least one useful microorganism selected from the group comprising Enterobactor cloacae subsp. cloacae Jordan Hormaeche and Edwards, subsp. nov. ATCC 13047, Enterobacter cloacae subsp. dissolvens ATCC 23373, Enterobacter cloacae Jordan Hormaeche and Edwards ATCC BAA-2806, Enterobacter cloacae Jordan Hormaeche and Edwards ATCC BAA-2468, Enterobacter cloacae Jordan Hormaeche and Edwards ATCC BAA-2341, Enterobacter cloacae subsp. cloacae Jordan Hormaeche and Edwards, subsp. nov. ATCC 23355, Enterobacter cloacae subsp. cloacae Jordan Hormaeche and Edwards, subsp. nov. ATCC 15337, Enterobacter cloacae Jordan Hormaeche and Edwards ATCC BAA-2273, Enterobacter cloacae Jordan Hormaeche and Edwards ATCC BAA-2080, Enterobacter cloacae subsp. cloacae Jordan Hormaeche and Edwards, subsp. nov. ATCC 961, Enterobacter cloacae subsp. cloacae Jordan Hormaeche and Edwards, subsp. nov. ATCC 15361, Enterobacter cloacae subsp. cloacae Jordan Hormaeche and Edwards, subsp. nov. ATCC 13599, Enterobacter cloacae subsp. cloacae Jordan Hormaeche and Edwards, subsp. nov. ATCC 35589, Enterobacter cloacae subsp. cloacae Jordan Hormaeche and Edwards, subsp. nov. ATCC 35930, Enterobacter cloacae subsp. cloacae Jordan Hormaeche and Edwards, subsp. nov. ATCC 27508, Enterobacter cloacae Jordan Hormaeche and Edwards ATCC BAA-2357, Enterobacter cloacae subsp. cloacae Jordan Hormaeche and Edwards, subsp. nov. ATCC 19336, Enterobacter cloacae subsp. cloacae Jordan Hormaeche and Edwards, subsp. nov. ATCC 29893, Enterobacter cloacae subsp. cloacae Jordan Hormaeche and Edwards, subsp. nov. ATCC 39978, Enterobacter cloacae subsp. cloacae Jordan Hormaeche and Edwards, subsp. nov. ATCC 35549, Enterobacter cloacae subsp. cloacae Jordan Hormaeche and Edwards, subsp. nov. ATCC 10699, Enterobacter cloacae subsp. cloacae Jordan Hormaeche and Edwards, subsp. nov. ATCC 7256, Enterobacter cloacae subsp. cloacae Jordan Hormaeche and Edwards, subsp. nov. ATCC 35929, Enterobacter cloacae subsp. cloacae Jordan Hormaeche and Edwards, subsp. nov. ATCC 27613, Enterobacter cloacae subsp. cloacae Jordan Hormaeche and Edwards, subsp. nov. ATCC 35587, Enterobacter cloacae subsp. cloacae Jordan Hormaeche and Edwards, subsp. nov. ATCC 35592, Enterobacter cloacae subsp. cloacae Jordan Hormaeche and Edwards, subsp. nov. ATCC 12666, Enterobacter cloacae subsp. cloacae Jordan Hormaeche and Edwards, subsp. nov. ATCC 700644, Enterobacter cloacae subsp. cloacae Jordan Hormaeche and Edwards, subsp. nov. ATCC BAA-1143, Enterobacter cloacae subsp. cloacae Jordan Hormaeche and Edwards, subsp. nov. ATCC 35030, Enterobacter cloacae subsp. cloacae Jordan Hormaeche and Edwards, subsp. nov. ATCC 35591, Enterobacter cloacae Jordan Hormaeche and Edwards ATCC BAA-2271, Enterobacter cloacae subsp. cloacae Jordan Hormaeche and Edwards, subsp. nov. ATCC 962, Enterobacter cloacae subsp. cloacae Jordan Hormaeche and Edwards, subsp. nov. ATCC 39979, Enterobacter cloacae subsp. cloacae Jordan Hormaeche and Edwards, subsp. nov. ATC 29006, Enterobacter cloacae subsp. cloacae Jordan Hormaeche and Edwards, subsp. nov. ATCC 700411, Enterobacter cloacae Jordan Hormaeche and Edwards ATCC BAA-2272, Enterobacter cloacae subsp. cloacae Jordan Hormaeche and Edwards, subsp. nov. ATCC 529, Enterobacter cloacae subsp. cloacae Jordan Hormaeche and Edwards, subsp. nov. ATCC 13047D-5, Enterobacter cloacae subsp. dissolvens ATCC 23373D-5, Enterobacter cloacae PTA-3882, Enterobacter hormaechei O'Hara et al. ATCC 700323, Enterobacter hormaechei O'Hara et al. ATC 700323, Cronobacter sakazakii ATCC 29004, Enterobacter cloacae subsp. Cloacae ATCC 700621, Enterobacter cloacae subsp. cloacae Jordan Hormaeche and Edwards, subsp. nov. ATCC 29941, Enterobacter cloacae subsp. cloacae Jordan Hormaeche and Edwards, subsp. nov. ATCC 43091, Klebsiella aerogenes Tindall et al. ATCC BAA-2358, Enterobacter amnigenus Izard et al. ATC 51816, Enterobacter cloacae subsp. cloacae Jordan Hormaeche and Edwards, subsp. nov. ATCC 35590, Enterobacter cloacae subsp. cloacae Jordan Hormaeche and Edwards, subsp. nov. ATCC 29005, Enterobacter cloacae subsp. cloacae Jordan Hormaeche and Edwards, subsp. nov. ATCC 29249, Klebsiella pneumoniae subsp. pneumoniae Schroeter Trevisan ATC 27889, Enterobacter cloacae subsp. cloacae Jordan Hormaeche and Edwards, subsp. nov. ATCC 222, Enterobacter cloacae subsp. cloacae Jordan Hormaeche and Edwards, subsp. nov. ATCC 33457, Enterobacter cloacae subsp. cloacae Jordan Hormaeche and Edwards, subsp. nov. ATCC 43560, Enterobacter cloacae subsp. cloacae Jordan Hormaeche and Edwards, subsp. nov. ATCC 49141, Enterobacter cloacae subsp. cloacae Jordan Hormaeche and Edwards, subsp. nov. ATC 35588, Enterobacter cloacae subsp. cloacae Jordan Hormaeche and Edwards, subsp. nov. ATCC 700258, Enterobacter amnigenus Izard et al. ATCC 33072, or Enterobacter kobei Kosako et al. ATCC BAA-260.

In some embodiments, the microorganism consortium in culture medium may optionally contain at least one useful microorganism selected from the group comprising Escherichia coli Migula Castellani and Chalmers ATCC 8739, Escherichia coli Migula Castellani and Chalmers ATCC 25922, Escherichia coli Migula Castellani and Chalmers ATCC 8739, Escherichia coli Migula Castellani and Chalmers ATCC CRM-8739, Escherichia coli Migula Castellani and Chalmers ATCC 25922 FDA strain Seattle 1946 [DSM 1103, NCIB 12210] Antigenic Properties Serotype 06, Biotype 1, Bacillus sp. ATCC BAA-1380, pJKK3-1 ATCC 37220, pBC16 ATCC 37338, Bacillus sp. ATCC 51912, ATCC 202074, ATCC 202075, or ATCC 202076.

As well, in some embodiments, the microorganism consortium in culture medium may optionally contain at least one useful microorganism selected from the group comprising Klebsiella aerogenes Tindall et al. ATCC 13048, Klebsiella aerogenes Tindall et al. ATCC 13048, Klebsiella aerogenes Tindall et al. ATCC 51697, Klebsiella aerogenes Tindall et al. ATCC 29008, Klebsiella aerogenes Tindall et al. ATCC 49701, Klebsiella aerogenes Tindall et al. ATCC 43175, Klebsiella aerogenes Tindall et al. ATCC 35028, Klebsiella aerogenes Tindall et al. ATC 29751, Klebsiella aerogenes Tindall et al. ATCC 15038, Klebsiella aerogenes Tindall et al. ATC BAA-2358, Klebsiella aerogenes Tindall et al. ATCC 49469, Klebsiella aerogenes Tindall et al. ATCC BAA-2347, Klebsiella aerogenes Tindall et al. ATCC 29010, Klebsiella aerogenes Tindall et al. ATCC BAA-2356, Klebsiella aerogenes Tindall et al. ATC 51342, Klebsiella aerogenes Tindall et al. ATCC 29007, Klebsiella aerogenes Tindall et al. ATCC 35029, Klebsiella aerogenes Tindall et al. ATCC 15038D-5, Klebsiella pneumoniae subsp. pneumoniae Schroeter Trevisan ATC 13882, Klebsiella pneumoniae subsp. pneumoniae Schroeter Trevisan ATCC 15380, Klebsiella oxytoca Flugge Lautrop ATC 15328, pSCH129 ATC 87423, Klebsiella pneumoniae subsp. pneumoniae Schroeter Trevisan ATCC 29519, Klebsiella pneumoniae subsp. pneumoniae Schroeter Trevisan ATCC 29517, Klebsiella aerogenes Tindall et al. ATCC 29009, Klebsiella pneumoniae subsp. pneumoniae Schroeter Trevisan ATCC 29518, Klebsiella pneumoniae subsp. pneumoniae Schroeter Trevisan ATCC 23357, Klebsiella oxytoca Flugge Lautrop ATCC 8724, Klebsiella pneumoniae subsp. pneumoniae Schroeter Trevisan ATC 13906, Klebsiella pneumoniae subsp. pneumoniae Schroeter Trevisan ATCC 13797, Klebsiella pneumoniae subsp. pneumoniae Schroeter Trevisan ATCC 23356, Klebsiella pneumoniae subsp. pneumoniae Schroeter Trevisan ATCC 25304, Klebsiella pneumoniae subsp. pneumoniae Schroeter Trevisan ATCC 9621, Klebsiella oxytoca Flugge Lautrop ATCC 12833, Klebsiella pneumoniae subsp. pneumoniae Schroeter Trevisan ATCC 25306, Klebsiella pneumoniae subsp. pneumoniae Schroeter Trevisan ATCC 12658, Klebsiella pneumoniae subsp. pneumoniae Schroeter Trevisan ATCC 21217, Klebsiella pneumoniae subsp. pneumoniae Schroeter Trevisan ATCC 21214, Klebsiella pneumoniae subsp. pneumoniae Schroeter Trevisan ATCC 21204, Klebsiella pneumoniae subsp. pneumoniae Schroeter Trevisan ATCC 25305, Klebsiella pneumoniae subsp. pneumoniae Schroeter Trevisan ATCC 211, Klebsiella pneumoniae subsp. pneumoniae Schroeter Trevisan ATC 12657, Klebsiella pneumoniae subsp. pneumoniae Schroeter Trevisan ATCC 21215, Klebsiella pneumoniae subsp. pneumoniae Schroeter Trevisan ATCC 27727, Klebsiella pneumoniae subsp. pneumoniae Schroeter Trevisan ATCC 27858, Klebsiella pneumoniae subsp. pneumoniae Schroeter Trevisan ATCC 25955, Klebsiella pneumoniae subsp. pneumoniae Schroeter Trevisan ATCC 8308, Klebsiella pneumoniae subsp. pneumoniae Schroeter Trevisan ATCC 21203, Klebsiella pneumoniae subsp. pneumoniae Schroeter Trevisan ATCC 21205, Klebsiella pneumoniae subsp. pneumoniae 21216, Klebsiella pneumoniae subsp. pneumoniae 21316, pMMB66EH ATCC 37620, pMMB67HE ATCC 37623, pJAK14 [JK388] ATCC 77289, pJAK16 [JK396] ATCC 77291, ATCC 77288, pJAK12 [JK386] ATCC 77287, pJAK17 [JK397] ATCC 77292, pMMB67EH ATCC 37622, pJAK15 [JK389] ATCC 77290, or pMMB66HE ATCC 37621.

In some embodiments, the microorganism consortium in culture medium may optionally contain at least one useful microorganism selected from the group comprising Rhodopseudomonas palustris Molisch van Niel ATCC 17001, Rhodopseudomonas palustris Molisch van Niel ATCC 33872, Rhodopseudomonas palustris Molisch van Niel ATCC 17005, Rhodopseudomonas palustris Molisch van Niel ATCC 17000, Rhodopseudomonas palustris Molisch van Niel ATCC BAA-1122, Rhodopseudomonas palustris Molisch van Niel ATCC 17010, Rhodopseudomonas palustris Molisch van Niel ATCC 17002, Rhodopseudomonas palustris Molisch van Niel ATCC 49781, Rhodopseudomonas palustris Molisch van Niel ATCC 17003, Rhodopseudomonas palustris Molisch van Niel ATCC 17004, Rhodopseudomonas palustris Molisch van Niel ATCC 51186, Rhodopseudomonas palustris Molisch van Niel ATCC 17006, Rhodopseudomonas palustris Molisch van Niel ATCC BAA-37, Rhodopseudomonas palustris Molisch van Niel ATCC 17008, Rhodopseudomonas palustris Molisch van Niel ATCC 17009, Rhodopseudomonas palustris Molisch van Niel ATCC BAA-98D-5, Rhodopseudomonas palustris Molisch van Niel ATCC BAA-1122D-5, Rhodopseudomonas palustris Molisch van Niel ATCC BAA-1123D-5, Rhodopseudomonas palustris Molisch van Niel ATCC BAA-98, Rhodopseudomonas palustris Molisch van Niel ATCC BAA-1123, Rhodopseudomonas palustris Molisch van Niel ATC BAA-1125, Rhodopseudomonas palustris Molisch van Niel ATCC 17007, Rhodopseudomonas palustris Molisch van Niel ATCC BAA-1124, or Rhodospirillum rubrum Esmarch Molisch ATCC 25852.

Additionally, in some embodiments, the microorganism consortium in culture medium may optionally contain at least one useful microorganism selected from the group comprising Rhodobacter sphaeroides van Niel Imhoff et al. ATC 17023, Rhodobacter sphaeroides van Niel Imhoff et al. ATCC 49419, Rhodobacter sphaeroides van Niel Imhoff et al. ATCC 17024, Rhodobacter sphaeroides van Niel Imhoff et al. ATCC 17026, Rhodobacter sphaeroides van Niel Imhoff et al. ATCC 21455, Rhodobacter sphaeroides van Niel Imhoff et al. ATCC BAA-808, Rhodobacter sphaeroides van Niel Imhoff et al. ATCC 35055, Rhodobacter sphaeroides van Niel Imhoff et al. ATCC 35053, Rhodobacter sphaeroides van Niel Imhoff et al. ATCC 35054, Rhodobacter sphaeroides van Niel Imhoff et al. ATCC 17023D-5, Rhodobacter sphaeroides van Niel Imhoff et al. ATCC BAA-808D-5, Rhodobacter sphaeroides van Niel Imhoff et al. ATCC 17025D-5, Rhodobacter sphaeroides van Niel Imhoff et al. ATCC 17029D-5, Rubrivivax sp. ATCC 55304, Rhodobacter sphaeroides van Niel Imhoff et al. ATC 17028, Rhodobacter sphaeroides van Niel Imhoff et al. ATCC 17029, Rhodobacter sphaeroides van Niel Imhoff et al. ATCC 17027, Rhodobacter sphaeroides van Niel Imhoff et al. ATCC 21286, Rhodobacter sphaeroides van Niel Imhoff et al. ATCC 33575, Paracoccus denitrificans Beijerinck and Minkman Davis emend. Rainey et al. ATCC 17741 Strain Designations: 381 [CIP 106306, CIP 106400, DSM 413, IAM 12479, ICPB 3979, IFO 16712, JCM 6892, LMD 22.21, LMG 4218, NCCB 22021, NCIB 11627, VKM B-1324], or Rhodobacter azotoformans ATC 17025.

Additionally, in some embodiments, the microorganism consortium in culture medium may optionally contain at least one useful microorganism selected from the group comprising Paenibacillus polymyxa Prazmowski Ash et al. ATCC 21830, Paenibacillus polymyxa Prazmowski Ash et al. ATCC 842, Paenibacillus polymyxa Prazmowski Ash et al. ATCC 7070, Paenibacillus polymyxa Prazmowski Ash et al. ATCC 43865, Paenibacillus polymyxa Prazmowski Ash et al. ATCC 12060 Strain Designations: CN 2222, Paenibacillus polymyxa Prazmowski Ash et al. ATCC 8525 Strain Designations: [NRS 813], Paenibacillus polymyxa Prazmowski Ash et al. ATCC 12321 Strain Designations: NRRL B-510 RHG, Paenibacillus polymyxa Prazmowski Ash et al. ATCC 8524 Strain Designations: [NRS 280; 8278], Paenibacillus polymyxa Prazmowski Ash et al. ATCC 7047 Strain Designations: NRS 251, Bacillus circulans Jordan ATCC 31228 Strain Designations: SC 10275, Paenibacillus polymyxa Prazmowski Ash et al. ATCC 8519 Strain Designations: [ATCC 9825, NRS 812], Paenibacillus polymyxa Prazmowski Ash et al. ATCC 8523 Strain Designations: [NRS 354], Paenibacillus polymyxa Prazmowski Ash et al. ATCC 39564 Strain Designations: 9A, Paenibacillus polymyxa Prazmowski Ash et al. ATCC 25901 Strain Designations: 63, Paenibacillus polymyxa Prazmowski Ash et al. ATCC 8526 Strain Designations: [NRS 297], Paenibacillus polymyxa Prazmowski Ash et al. ATCC 7070 Strain Designations: [Difco Labs. 8277, N. R. Smith NRS 279, Vitek #200115], Paenibacillus polymyxa Prazmowski Ash et al. ATCC 10401 Strain Designations: [BUCSAV 163, Boots 163, CCM 1460, CCM 1461, CCM 1465, NCDO 731, NCIB 8094, NCTC 1380, NRS 2010], Paenibacillus polymyxa Prazmowski Ash et al. ATCC 27955 Strain Designations: M4, Paenibacillus polymyxa Prazmowski Ash et al. ATCC 21551 Strain Designations: FH-K 890/3, Paenibacillus polymyxa Prazmowski Ash et al. ATCC 31037 Strain Designations: BN-109 [FERM-P 2129], Paenibacillus polymyxa Prazmowski Ash et al. ATCC 12712 Strain Designations: PRL B505, Paenibacillus polymyxa Prazmowski Ash et al. ATCC 21993 Strain Designations: 3-8 [FERM-P 412],

As well, in some embodiments, the microorganism consortium in culture medium may optionally contain at least one useful microorganism selected from the group comprising Paenibacillus chitinolyticus NRRL B-23119, Paenibacillus macerans Schardinger Ash et al. ATCC 8244 Strain Designations: NRS 888 [NCIB 9368, NCTC 6355], Paenibacillus macerans Schardinger Ash et al. ATCC 8509 Strain Designations: 18 [NRS 1095, Vitek #200237], Paenibacillus macerans Schardinger Ash et al. ATCC 7069 Strain Designations: NRS 277 [8275], Paenibacillus macerans Schardinger Ash et al. ATCC 8510 Strain Designations: 2037 [NRS 1096], Paenibacillus macerans Schardinger Ash et al. ATCC 8517 Strain Designations: 583 [NRS 1101], Paenibacillus macerans Schardinger Ash et al. ATCC 843 Strain Designations: [NRS 1093], Paenibacillus macerans Schardinger Ash et al. ATCC 8514 Strain Designations: 588 [NRS 1098], Paenibacillus macerans Schardinger Ash et al. ATCC 7048 Strain Designations: NRS 649 [ATCC 8512], Paenibacillus macerans Schardinger Ash et al. ATCC 8513 Strain Designations: 577 [NRS 1097], Paenibacillus macerans Schardinger Ash et al. ATCC 8509 Strain Designations: 18 [NRS 1095, Vitek #200237], Paenibacillus macerans Schardinger Ash et al. ATCC 8516 Strain Designations: 585 [NRS 1100], Paenibacillus macerans Schardinger Ash et al. ATCC 7068 Strain Designations: NRS 278 [8276], Paenibacillus macerans Schardinger Ash et al. ATCC 8518 Strain Designations: [NRS 646], Paenibacillus macerans Schardinger Ash et al. ATCC 8515 Strain Designations: 573 [NRS 1099], Paenibacillus macerans Pma 007 BR-97 202152, or Paenibacillus graminis Berge et al. ATCC 49035 Strain Designations: NRRL B-390 [NRS 373].

In one or more embodiments, the at least one useful microorganism may be selected from a group comprising Pseudomonas putida Trevisan Migula ATCC 15070, Pseudomonas putida NRRL B-8, Pseudomonas putida NRRL B-13, Pseudomonas putida NRRL B-21, Pseudomonas putida NRRL B-22, Pseudomonas putida NRRL B-251, Pseudomonas putida NRRL B-252, Pseudomonas putida NRRL B-254, Pseudomonas putida NRRL B-723, Pseudomonas putida NRRL B-805, Pseudomonas putida NRRL B-993, Pseudomonas putida NRRL B-1023, Pseudomonas putida NRRL B-1245, Pseudomonas putida NRRL B-1486, Pseudomonas putida NRRL B-1595, Pseudomonas putida NRRL B-2084, Pseudomonas putida NRRL B-2267, Pseudomonas putida NRRL B-2459, Pseudomonas putida NRRL B-2914, Pseudomonas putida NRRL B-4067, Pseudomonas putida NRRL B-1468, Pseudomonas putida NRRL B-14875, Pseudomonas putida NRRL B-14878, Pseudomonas putida NRRL B-14887, Pseudomonas putida NRRL B-14888, Pseudomonas putida NRRL B-14938, Pseudomonas putida NRRL B-23263, Pseudomonas putida NRRL B-41390, Pseudomonas putida NRRL B-2084 Pseudomonas striata, or Pseudomonas putida NRRL B-2914 Pseudomonas striata.

In one or more embodiments, the at least one useful microorganism may be selected from a group comprising Pseudomonas fluorescens Migula ATCC 13525 Strain Designations: NCTC 10038 [28/5, CCEB 546, DSM 50090, NCIB 9046, NCPPB 1964, PJ239, R. Hugh 818, R.Y. Stanier 192, Biotype A], Pseudomonas fluorescens Migula ATCC 17400 Strain Designations: 18 [IFO 15833, WRRL P-7], Pseudomonas fluorescens Migula ATCC 17556 Strain Designations: 188 [NCPPB 316 Pseudomonas marginata, PJ 160], Pseudomonas fluorescens Migula ATCC 17550 Strain Designations: 182 [IFO 3081, PJ 73], Pseudomonas fluorescens Migula ATCC 17555 Strain Designations: 187 [NCPPB 263 Pseudomonas angulata, PJ 139], Pseudomonas fluorescens Migula ATCC BAA-1781 Strain Designations: CNG89, Pseudomonas fluorescens Migula ATCC 31732, Pseudomonas fluorescens Migula ATCC 53795, Pseudomonas fluorescens Migula ATCC 13459 Strain Designations: PW2 [ICMP 3966, NCPPB 967, NRRL B-899], Pseudomonas fluorescens Migula ATCC 15917 Strain Designations: IAM 1055 [AHH-23], Pseudomonas fluorescens Migula ATCC 43203, Pseudomonas fluorescens Migula ATCC 33512 Strain Designations: 72, Pseudomonas fluorescens Migula ATCC 15456 Strain Designations: CCEB 488-A [BU 140], Pseudomonas fluorescens Migula ATCC 11253 Strain Designations: [NRRL B-1244], Pseudomonas fluorescens Migula ATCC 39502 Strain Designations: SC 12936, Pseudomonas fluorescens Migula ATCC 17554 Strain Designations: 186 [IFO 15829, PJ 79], Pseudomonas fluorescens Migula ATCC 700281 Strain Designations: H13, Pseudomonas fluorescens Migula ATCC 21541 Strain Designations: 30-21, Pseudomonas fluorescens Migula Strain Designations: 1291 [ATCC 17458, IFO 15837, NCIB 8917; LA, NRRL B-1864, R.Y. Stanier 83], Pseudomonas fluorescens Migula ATCC 17513 Strain Designations: 143 [IFO 15836, PL], Pseudomonas fluorescens Migula ATCC 17482 Strain Designations: 108 [52-22, IFO 15832], Pseudomonas fluorescens Migula ATCC 31950 Strain Designations: A505 [A5-05-1], Pseudomonas fluorescens Migula ATCC 12843 Strain Designations: NCIB 8866 strain CO2, Pseudomonas fluorescens Migula ATCC 27663 Strain Designations: PW, Pseudomonas fluorescens Migula ATCC 17574 Strain Designations: 207 [PJ 693], Pseudomonas fluorescens Migula ATCC 15453 Strain Designations: 52-1C, Pseudomonas fluorescens Migula ATCC 49667 Strain Designations: F-12, Pseudomonas fluorescens Migula ATCC 17467 Strain Designations: 93 [TR-10], Pseudomonas fluorescens Migula ATCC 17569 Strain Designations: 202 [PJ 372], Pseudomonas fluorescens Migula ATCC 15553 Strain Designations: CCEB 553 [IEM 15/47], Pseudomonas fluorescens Migula ATCC 31419 Strain Designations: IAM-1126 [43F], Pseudomonas fluorescens Migula ATCC 17565 Strain Designations: 198 [PJ 302], Pseudomonas fluorescens Migula ATCC 17575 Strain Designations: 208 [PJ 722], Pseudomonas fluorescens Migula ATCC 49270 Strain Designations: F 1847 [CDC-EB], Pseudomonas fluorescens Migula ATCC 17579 Strain Designations: 212 [PJ 832], Pseudomonas fluorescens Migula ATCC 17563 Strain Designations: 196 [PJ 288], Pseudomonas fluorescens Migula ATCC 13430 Strain Designations: Pyrrolidine, Pseudomonas fluorescens Migula ATCC 17557 Strain Designations: 189 [PJ227; 1208], Pseudomonas fluorescens Migula ATCC 17518 Strain Designations: 149 [2-40-40, IFO 15838], Pseudomonas fluorescens Migula ATCC 13475, Pseudomonas fluorescens Migula ATCC 17570 Strain Designations: 203 [PJ 376], Pseudomonas fluorescens Migula ATCC 949 Strain Designations: 1062, Pseudomonas fluorescens Migula ATCC 23728, Pseudomonas fluorescens Migula ATCC 17573 Strain Designations: 206 [PJ 692], Pseudomonas fluorescens Migula ATCC 17559 Strain Designations: 191 [IFO 15834, PJ 236; 22/1], Pseudomonas fluorescens Migula ATCC 17561 Strain Designations: 194 [Klinge R-60, PJ 253], Pseudomonas fluorescens Migula ATCC 35858 Strain Designations: NRRL B-4290, Pseudomonas fluorescens Migula ATCC 17582 Strain Designations: 215 [PJ 849], Pseudomonas fluorescens Migula ATCC 6972 Strain Designations: NCTC 2583 [NCIB 8194], Pseudomonas fluorescens Migula ATCC 17553 Strain Designations: 185 [W2 L-1], Pseudomonas fluorescens Migula ATCC 17571 Strain Designations: 204 [IFO 15835, PJ 682], Pseudomonas fluorescens Migula ATCC 55129 Strain Designations: SC 15208, Pseudomonas fluorescens bacteriophage phi-S1 ATCC 27663-B1 Strain Designations: Phi-S1, Pseudomonas fluorescens Migula ATCC 700830, Pseudomonas fluorescens Migula ATCC 53794 Strain Designations: PRA25, Pseudomonas fluorescens Migula ATCC 17552 Strain Designations: 184 [IFO 15830], Pseudomonas fluorescens Migula ATCC 17572 Strain Designations: 205 [PJ 686], Pseudomonas fluorescens Migula ATCC 23611, Pseudomonas fluorescens Migula ATCC 31125 Strain Designations: P-2563 [FERM-P 2894, IFO 13658], Pseudomonas fluorescens Migula ATCC 25289 Strain Designations: PYR, Pseudomonas fluorescens Migula ATCC 17634 Strain Designations: 267 [B-9], Pseudomonas fluorescens Migula ATCC 55241 Strain Designations: BNL-WVC, Pseudomonas fluorescens Migula ATCC 11150 Strain Designations: [NCIB 8286], Pseudomonas fluorescens Migula ATCC 49656, Pseudomonas fluorescens Migula ATCC 17568 Strain Designations: 201 [PJ 368], Pseudomonas fluorescens Migula ATCC 15916 Strain Designations: IAM 1008 [AHH-27], Pseudomonas fluorescens Migula ATCC 948 Strain Designations: 1013 [ATCC 11251, CCEB 295, IFO 3903, Jessen PJ 70, KY 3975, NCIB 9493, NRRL B-10, Stanier 181], Pseudomonas fluorescens Migula ATCC 14150 Strain Designations: NRRL B-1603 [6, IFO 15840], Pseudomonas fluorescens Migula ATCC 53859 Strain Designations: 6133D02, Pseudomonas fluorescens 53958, Pseudomonas fluorescens CGA 270294 55174, Pseudomonas fluorescens CGA 270293 55175, Pseudomonas fluorescens CGA 281836 55168, Pseudomonas fluorescens biovar I: MB214 PTA-7840, Pseudomonas fluorescens 21256, Pseudomonas fluorescens, Pseudomonas fluorescens PTA-3749, Pseudomonas sp. formerly Pseudomonas fluorescens 55084, Pseudomonas fluorescens CGA 266446 55171, Pseudomonas fluorescens biovar I: MB101 PTA-7841, Pseudomonas fluorescens 53860, Pseudomonas putida Trevisan Migula ATCC 12633, Strain Designations: [A.3.12, ATCC 23467, NCIB 9494, NCTC 10936, R.Y. Stanier 90], Pseudomonas aeruginosa Schroeter Migula ATCC 9721 Strain Designations: NRS 112 [NRRL B-7, R. Hugh 814], Pseudomonas protegens Ramette et al. ATCC BAA-477 Strain Designations: Pf-5, Pseudomonas brenneri ATCC 49642 Strain Designations: P17, Pseudomonas protegens Ramette et al. ATCC 17386 Strain Designations: 1 [IFO 15842], Pseudomonas chlororaphis Guignard and Sauvageau Bergey et al. ATCC 17809 Strain Designations: 388 [NRRL B-1854], Pseudomonas putida Trevisan Migula ATCC 31483 Strain Designations: 3P, Pseudomonas putida Trevisan Migula ATCC 11250 Strain Designations: Tr. 23 [NCIB 10098], Pseudomonas aeruginosa Schroeter Migula ATCC 31156 Strain Designations: KY 3955 [FERM-P 2611], Pseudomonas chlororaphis Guignard and Sauvageau Bergey et al. ATCC 17812 Strain Designations: 391 [NRRL B-1869], pSCH5102 ATCC 87426, Pseudomonas chlororaphis Guignard and Sauvageau Bergey et al. ATCC 17813 Strain Designations: 392 [Lysenko 4, NRRL B-2075], Burkholderia cepacia Palleroni and Holmes Yabuuchi et al. ATCC 29424 (Pseudomonas fluorescens Migula) Strain Designations: PHK, Pseudomonas putida Trevisan Migula ATCC 12842 Strain Designations: NCIB 8865 strain C01, Pseudomonas aeruginosa Schroeter Migula ATCC 12121 Strain Designations: P-6, Pseudomonas aeruginosa Schroeter Migula ATCC 9721 Strain Designations: NRS 112 [NRRL B-7, R. Hugh 814], Pseudomonas chlororaphis Guignard and Sauvageau Bergey et al. ATCC 17810 Strain Designations: 389 [NRRL B-1854], Pseudomonas migulae ATCC 39005 Strain Designations: PMW6 [NCIB 11615], Pseudomonas aeruginosa Schroeter Migula ATCC 8689 Strain Designations: 668 [R. Hugh 810, USDA 604], Pseudomonas aeruginosa Schroeter Migula ATCC 10796 Strain Designations: 68, Pseudomonas putida Trevisan Migula ATCC 11172 Strain Designations: NCIB 8251 [DSM 6521], Pseudomonas aeruginosa Schroeter Migula ATCC 142 Strain Designations: AMC [NRRL B-12], Pseudomonas sp. ATCC 49323 Strain Designations: NCIB 10586, Pseudomonas chlororaphis Guignard and Sauvageau Bergey et al. ATCC 17814 Strain Designations: 393 [NRRL B-1632; 261], Pseudomonas chlororaphis Guignard and Sauvageau Bergey et al. ATCC 17411 Strain Designations: 32 [NCIB 9402, NRRL B-977; NCTC 7357], Pseudomonas chlororaphis Guignard and Sauvageau Bergey et al. ATCC 17419 Strain Designations: 41, Pseudomonas fluorescens Migula ATCC Strain Designations: 197 [PJ 290], Pseudomonas fluorescens Migula ATCC 17397 Strain Designations: 12 [ATCC 25323, NIH 11, den Dooren de Jong 216], Pseudomonas chlororaphis Guignard and Sauvageau Bergey et al. ATCC 17415 Strain Designations: 36, Pseudomonas fluorescens Migula ATCC 17816 Strain Designations: 401 [C71A, IFO 15831, PJ 187], Pseudomonas fluorescens Migula ATCC 17638 Strain Designations: 271 [B-1612], Pseudomonas fluorescens Migula ATCC 31086 Strain Designations: D946-B83 [BU 2183, FERM-P 3328], Pseudomonas fluorescens Migula ATCC 49036 Strain Designations: A1 [IFO 15839], Pseudomonas chlororaphis Guignard and Sauvageau Bergey et al. ATCC 17811 Strain Designations: 390 [NRRL B-1095], Pseudomonas aeruginosa Schroeter Migula ATCC 8707 Strain Designations: 604 [R. Hugh 811], Pseudomonas fluorescens Migula ATCC 17583 Strain Designations: 216 [PJ 885], Pseudomonas chlororaphis Guignard and Sauvageau Bergey et al. ATCC 17461 Strain Designations: 86, Pseudomonas fluorescens Migula ATCC 49271 Strain Designations: F 1848 [CDC 93], Pseudomonas chlororaphis Guignard and Sauvageau Bergey et al. ATCC 17414 Strain Designations: 35 [NRRL B-1541], Pseudomonas fluorescens Migula ATCC 17926 Strain Designations: NRRL B-3178 [4, IFO 15841], Pseudomonas fluorescens Migula ATCC 33231 Strain Designations: B69, Pseudomonas mosselii Dabboussi et al. ATCC 49838 Strain Designations: AmMS 257, Pseudomonas fluorescens Migula ATCC 21637 Strain Designations: [IFO 3081], Pseudomonas fluorescens Migula ATCC 31948 Strain Designations: A506 [A5-06], Pseudomonas protegens Ramette et al. ATCC BAA-477D-5 Strain Designations: Genomic DNA from Pseudomonas protegens strain Pf-5 [ATCC BAA-477], ATCC 21539, ATCC 31951, ATCC 55034, Pseudomonas synxantha Ehrenberg Holland ATCC 9890 Strain Designations: [CCEB 293, NRRL B-780], Pseudomonas chlororaphis Guignard and Sauvageau Bergey et al. ATCC 17413 Strain Designations: 34 [NRRL B-1098], Aspergillus niger van Tieghem ATCC 16888, Penicillium roqueforti Thom ATCC 10110, Herminiimonas sp. ATCC 49643 Strain Designations: NOX, Rhizopus stolonifer Ehrenberg: Fries Lind ATCC 14037, or MA1-6 ATCC CRL-1783.

In one or more embodiments, the at least one useful microorganism may be any strain of Pseudomonas calcis.

In one or more embodiments, the at least one useful microorganism may be selected from the group comprising of Acrothcium, Actinomyces, Alternaria, Arthrobotrys, Aspergillus, Candida, Cephalosporium, Cladosporium, Curvularia, Cunninghamella, Chaetomium, Cryptococcus, Debaryomyces, Fusarium, Glomus, Helminthosporium, Hansenula, Klockera, Micromonospora, Mortierella, Myrothecium, Oidiodendron, Paecilomyces, Penicillium, Phoma, Pichia, Populospora, Rhizoctonia, Rhizopus, Rhodotorula, Saccharomyces, Schizosaccharomyces, Schwanniomyces, Sclerotium, Torula, Trichoderma, or Yarrowia.

In another embodiment, the at least one useful microorganism may be selected from a group comprising Mortierella alliacea, Mortierella alpina, M. polycephala, Mortierella elongata, Mortierella spinosa, Mortierella gamsii, Mortierella isabellina, Mortierella humilis, or Mortierella reticulata.

In one or more embodiments, at least one useful microorganism may be selected from a group comprising Aspergillus rugulosus MB 277104, Aspergillus rugulosa ATCC 16820, Aspergillus rugulosa ATCC 32659, Aspergillus rugulosa ATCC 32623, Aspergillus rugulosa ATCC 32660, Aspergillus rugulosa ATCC 32661, Aspergillus rugulosa ATCC 58398, Aspergillus rugulosa ATCC 64625, Aspergillus rugulosa ATCC 16381, Aspergillus rugulosa ATCC 64624, Aspergillus rugulosa ATCC 22283, Aspergillus niger, and Aspergillus oryzae.

In another embodiment, the at least one useful microorganism may be selected from a group comprising Penicillium bilaiae ATCC 18309, Penicillium bilaiae ATCC 20851, Penicillium bilaiae ATCC 22348, Penicillium bilaiae NRRL 50162, Penicillium bilaiae NRRL 50169, Penicillium bilaiae NRRL 50776, Penicillium bilaiae NRRL 50777, Penicillium bilaiae NRRL 50778, Penicillium bilaiae NRRL 50777, Penicillium bilaiae NRRL 50778, Penicillium bilaiae NRRL 50779, Penicillium bilaiae NRRL 50780, Penicillium bilaiae NRRL 50781, Penicillium bilaiae NRRL 50782, Penicillium bilaiae NRRL 50783, Penicillium bilaiae NRRL 50784, Penicillium bilaiae NRRL 50785, Penicillium bilaiae NRRL 50786, Penicillium bilaiae NRRL 50787, Penicillium bilaiae NRRL 50788, Penicillium bilaiae RS7B-SD1, Penicillium brevicompactum AgRF18, Penicillium canescens ATCC 10419, Penicillium expansum ATCC 24692, Penicillium expansum YT02, Penicillium fellatanum ATCC 48694, Penicillium gaestrivorus NRRL 50170, Penicillium glabrum DAOM 239074, Penicillium glabrum CBS 229.28, Penicillium janthinellum ATCC 10455, Penicillium lanosocoeruleum ATCC 48919, Penicillium radicum ATCC 201836, Penicillium radicum FRR 4717, Penicillium radicum FRR 4719, Penicillium radicum N93/47267 and/or Penicillium raistrickii ATCC 10490, or Penicillium purpurogenum var. Rubrisclerotium MB 124011.

In some embodiments, the microorganism consortium in culture medium contains a yeast microorganism. Yeast microorganisms include genera and species within the Ascomycota phylum, including true yeasts and fission yeasts. Preferred yeast microorganisms may include Saccharomyces genus and combinations thereof. Examples of useful yeast include for example Saccharomyces cerevisiae. In one embodiment, a microorganism consortium in culture medium contains a Saccharomyces cerevisiae. In a more specific embodiment, the subject yeast is Saccharomyces cerevisiae CNCM 1-3060, Saccharomyces cerevisiae NCYC R397, Saccharomyces cerevisiae CNCM 1-3399, Saccharomyces cerevisiae NCYC R646, Saccharomyces cerevisiae CBS 493.9, Saccharomyces cerevisiae CNCM 1-1077, Saccharomyces cerevisiae NCYC Sc 47, Saccharomyces cerevisiae CNCM 1-4407, Saccharomyces cerevisiae MUCL 39885, Saccharomyces cerevisiae NCYC R404, Saccharomyces cerevisiae NCYC R404, Saccharomyces cerevisiae PCM KKP 2059p, or Saccharomyces cerevisiae CNCM 1-1079.

Additionally, the at least one yeast may be selected from a group comprising Acrothcium robustum ATCC 10715, Acrothecium capsici ATCC 10714, Candida montana, Candida etchellsii ATCC 60167, Candida versatilis ATCC 60121 or Candida robusta Diddens et Lodder.

In one embodiment, the yeast is a species of Saccharomyces. In a more specific embodiment, the yeast is Saccharomyces cerevisiae. In a still more specific embodiment, the yeast is a specific strain of Saccharomyces cerevisiae that is generally recognized as safe for ingestion or approved for use in animal feed or human food, such as e.g., Saccharomyces cerevisiae NCYC Sc47, Saccharomyces cerevisiae PCM KKP 2059p, or Saccharomyces cerevisiae IFO 0203.

In one embodiment, the non-pathogenic gram-positive Bacilli bacteria capable of fermentation is one or more of Bifidobacterium spp., Lactobacillus spp., Lactococcus spp., Carnobacterium spp., Streptococcus spp., and Bacillus spp. In a more specific embodiment, the non-pathogenic gram-positive Bacilli bacteria capable of fermentation is one or more of Bifidobacterium animalis, Lactobacillus acidophilus, Bacillus subtilus, Lactococcus lactis, Lactobacillus casei, Lactobacillus plantarum, Carnobacterium divergens, and Lactobacillus rhamnosus.

In a particular embodiment, the subject non-pathogenic gram-positive Bacilli bacteria capable of fermentation comprises Bifidobacterium animalis DSM 16284, Lactobacillus acidophilus CECT 4529, and Bacillus subtilus ATCC PTA 6737. In another particular embodiment, the subject non-pathogenic gram-positive Bacilli bacteria capable of fermentation comprises Lactococcus lactis PCM B/00039, Lactobacillus casei PCM B/00080, Lactobacillus plantarum PCM B/00081, and Carnobacterium divergens PCM KKP 2012p. In yet another particular embodiment, the subject non-pathogenic gram-positive Bacilli bacteria capable of fermentation comprises Lactobacillus rhamnosus ATCC 7469.

In some embodiments, the first additive may comprise fulvic acid, water, and in some embodiments, other ingredients. In some embodiments, the first additive comprises up to 20% fulvic acid, up to 70% kelp or kelp-derived material, and up to 80% water.

Additionally, in some embodiments, the first additive may comprise a chelating agent. In some embodiments, the first additive may comprise an organic acid chelating agent. In some embodiments, the chelating agent of the first additive may comprise fulvic acid, humic acid, propionic acid, citric acid, homo citric acid, ascorbic acid, lactic acid, oxalic acid, vanillic acid, gallic acid, malate, gluconic acid, glucoheptonic acid, 2,3-dihydroxybenzoic acid, 1,4,7-tetra azacyclododecane 1,4,7,10-tetraacetic acid, ethylenediaminetetraacetic acid, N-(hydroxyethyl)ethylenediamine-triacetic acid, diethylene-triamine penta acetic acid, ethylenediamine-N,N′-bis(2-hydroxyphenyl)acetic acid, ethylenediamine-N,N′-bis(2-metoxyphenyl)acetic acid, ethylenediamine-N,N′-bis(2-hydroxy-4-methylphenyl)acetic acid, ethylenediamine-di-o-hydroxyphenylacetic acid, ethylene-diamine-di-(5-carboxy-2-hydroxyphenyl)acetic acid, ethylenediamine-N,N′-bis(4-hydroxyphenyl)acetic acid, ethylenediamine-N,N′-bis(2-hydroxy-5-methylphenyl)acetic acid, ethylenediamine-N,N′-bis(2-hydroxy-5-carboxyphenyl)acetic acid, ethylenediamine-N,N′-bis(2-hydroxy-5-sulfonylphenyl)acetic acid, ethylenediamine-N, N′-disuccinic acid, ethylenediamine-di-(o-hydroxy-p-methylphenyl) acetic acid, ethylenediamine-N,N′-bis(2-hydroxyphenylacetic acid), ethylenediamine-N,N-tetraacetic acid, N-carboxymethyliminobis(ethylenenitrilo)tetraacetic acid, ethylenediamine-N-(2-hydroxyphenylaceticacid)-N′-(4-hydroxyphenylacetic acid), N-(2-hydroxyethyl)ethylenediamine-N,N′,N′-triacetic acid nitrilotriacetic acid, ethylenediamine-N,N′-bis(2-hydroxy-6-methylphenylacetic acid), ethylenediamine-N,N′-bis(2-hydroxy-5-sulfophenylacetic acid) and oxoacetic acid polymer with 1,2-ethanediamine and 4-hydroxybenzenesulfonic acid ethylenediamine-N,N′-bis(5-carboxy-2-hydroxyphenylacetic acid), ethylenediamine-N,N′-bis(4-carboxy-2-hydroxyphenylacetic acid), or ethylenediamine-N,N′-bis(2-carboxy-5-hydroxyphenylacetic acid).

The at least one chelating agent may also comprise a siderophore. In some embodiments, the siderophore may be a microbial siderophore, a synthetic siderophore, or a phytosiderophore. In some embodiments, the siderophore may comprise, without limitation, a hydroxamate siderophore, catecholate siderophore, or carboxylate siderophore. Non-limiting examples of siderophores include enterobactin, azotochelin, azotobactin, rhizobactin, ferrioxamine B, ferrioxamine G, ferrioxamine E, ferricrocin, ferrichrome, mugineic acid, 2′-deoxymugineic acid, avenic acid A, distichonic acid, desferrioxamine B, as well as any snychobactin, petrobactin, aerobactin, marinobactin, taiwachelin, pyoverdine, loihichelin, aquachelin, alterobactin, ochrobactin, parabactin, or other siderophore.

As well, the chelating agent may comprise a dicarboxylic acid having at least one β-hydroxyl group, a tricarboxylic acid having at least one β-hydroxyl group, a phenolic acid having at least one ortho-hydroxyl group, an organic acid configured to chelate cobalt, an organic acid configured to chelate iron, an organic acid configured to chelate selenium, an organic acid configured to chelate silicon, an organic acid configured to chelate manganese, an organic acid configured to chelate copper, an organic acid configured to chelate zinc, an organic acid configured to chelate molybdenum, and an organic acid configured to chelate boron or any other micronutrient.

The at least one chelating agent of the first additive may comprise one or more amino acids. In some embodiments, the amino acid chelating agent may comprise one or more of alanine, arginine, asparagine, aspartic acid, cysteine, glutamic acid, glutamine, glycine, histidine, hydroxyproline, isoleucine, leucine, lysine, methionine, phenylalanine, proline, pyroglutamic acid, serine, threonine, tryptophan, tyrosine, and valine.

The phrase “chelated metal” may refer to a central metal ion attached to a chelating agent in a chemical complex. “Central metal ion” may mean any ion, any cation, which in some embodiments may comprise the cation of iron, manganese, zinc, copper, magnesium, nickel, boron, or molybdenum. “Chelating agent” may mean any chemical compound with a structure permitting the attachment of two or more donor atoms to the same central metal ion simultaneously to produce one or more rings.

In some embodiments, the second additive may comprise, at least, a nitrogen source, a potassium source, a chelated metal, and a salt.

In some embodiments, the nitrogen source may comprise a peptide, a polypeptide, manure, any collagen-derived protein hydrolysate, soybeans or soybean hydrolysate, peas or pea protein hydrolysate, whey or whey protein hydrolysate, rice or rice grain protein hydrolysate, alfalfa or alfalfa protein hydrolysate, legume seeds or legume seed protein hydrolysate, corn or corn protein hydrolysate, byproducts of corn wet-milling, any other plant-derived protein hydrolysate, casein or casein-derived protein hydrolysate, yeast extract, tryptone, beef extract, peptone, potassium nitrate, ammonium nitrate, ammonium chloride, ammonium sulfate, ammonium phosphate, ammonia, distiller's dried grains with solubles (DDGS), urea, any protein hydrolysate, carbohydrates, mineral elements, phenols, phytohormones, bioactive peptides acting as signaling molecules, an amino acid (such as but not limited to glycine, proline, hydroxylysine, hydroxyproline or aspartic and glutamic acids), peptides involved in metal chelating and binding (glutathione and phytochelatins), root hair promoting peptides, and other organic compounds. Additives having one or more of the above-described properties not otherwise named are contemplated as nitrogen sources.

The amount of nitrogen source varies according to the nitrogen source, typically between 0.1 to 30 grams per liter of medium. The weight fraction of the nitrogen source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In some embodiments, the potassium source of the second additive may comprise potassium oxide, potassium citrate, potassium sulfate, monopotassium phosphate, potassium dihydrogen phosphate, potassium nitrate, potassium chloride, or ammonium phosphate.

In some embodiments, the chelated metal of the second additive may comprise iron EDDHA, manganese EDTA, or zinc EDTA.

In some embodiments, the salt of the second additive may comprise boric acid, disodium octaborate tetrahydrate, sodium molybdate, ferrous sulfate, manganese sulfate, or zinc sulfate. By way of illustration, additional examples of suitable inorganic salts include, without limitation, potassium dihydrogen phosphate, dipotassium hydrogen phosphate, disodium hydrogen phosphate, magnesium sulfate, magnesium chloride, ferric sulfate, ferric chloride, ferrous chloride, manganous sulfate, manganous chloride, zinc chloride, cupric sulfate, calcium chloride, sodium chloride, calcium carbonate, sodium carbonate, and combinations thereof. The weight fraction of the inorganic salt in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In some embodiments, the second additive may comprise any other organism-derived fertilizer such as fertilizers derived from but not limited to human or animal waste and vegetable matter, as well as any organism-derived matter in its natural form. Examples include but are not limited to peat, manure, animal urine, raw sewage, bat guano, bird guano, treated sewage, sewage sludge, compost, human excrement or urine, animal by-products such as bone, tissue, skin, feathers, blood, hides, hoofs, or horns, animal waste or by-products mixed with sawdust or other naturally-occurring fibers, and other organic fertilizers.

In some embodiments, the second additive may comprise protein hydrolysate, potassium citrate, disodium octaborate tetrahydrate, ferrous sulfate, manganese sulfate, and zinc sulfate. In some embodiments, the second additive may comprise 2-25% (w/w) protein hydrolysate, 5-25% (w/w) potassium citrate, 1-10% (w/w) disodium octaborate tetrahydrate, 1-10% (w/w) ferrous sulfate, 1-10% (w/w) manganese sulfate, and 1-10% zinc sulfate. In some embodiments, the second additive may comprise 2-15% (w/w) protein hydrolysate, 5-10% (w/w) potassium citrate, 1-5% (w/w) disodium octaborate tetrahydrate, 1-5% (w/w) ferrous sulfate, 1-5% (w/w) manganese sulfate, and 1-5% zinc sulfate. In some embodiments, the second additive may comprise 5.0% (w/w) protein hydrolysate, 8.0% (w/w) potassium citrate, 0.6% (w/w) disodium octaborate tetrahydrate, 1.6% (w/w) ferrous sulfate, 1.6% (w/w) manganese sulfate, and 1.6% zinc sulfate.

In some embodiments, the second additive may comprise at least 5.0% (w/w) water soluble nitrogen, 8.0% (w/w) soluble potash, 0.6% (w/w) boron, 1.6% (w/w) iron, 1.6% (w/w) manganese, and 1.6% zinc. Moreover, in some embodiments, the second additive may comprise at least 5.0% (w/w) protein hydrolysate, 8.0% (w/w) potassium citrate, 0.6% (w/w) disodium octaborate tetrahydrate, 1.6% (w/w) ferrous sulfate, 1.6% (w/w) manganese sulfate, and 1.6% zinc sulfate.

In some embodiments, the water may comprise any water whether modified or unmodified. In some embodiments, the water may comprise chlorine-free water. Chlorine-free water (or “dechlorinated” water) may be obtained by exposing the water to air and allowing the chlorine to evaporate, by carbon-filtering the water, or any by other known means of dechlorinating water. In some embodiments, however, the water may comprise filtered water, twice filtered water, treated water, water having additives, filtered sewage, runoff water, greywater, tap water, well water, carbonated water, seawater, lakewater, pondwater, rainwater, any of which may be captured, redirected, modified, or produced by known means.

In at least one embodiment, the biofertilizer composition may further comprise a carbon source. In some embodiments, the carbon source may contain a carbohydrate, such as glucose, starch, cellulose, fructose, sucrose, or the like. In one embodiment, the carbon source is plant material such as silage. In another embodiment, the carbon source is a plant material from the grass family Poaceae. In some embodiments, the carbon source is relatively unrefined plant material, such as silage, stover, chaff, grass, stalks, leaves, and the like. In other embodiments, the carbon source comprises a more refined plant material, such as flour, syrup, molasses, or the like. In another embodiment, the carbon source comprises purified or semi-purified organic molecules, such as protein, fat, fatty acids, carbohydrates, or the like. In a more specific embodiment, the carbon source comprises a bran from grain, such as, e.g., rice bran, or a syrup or molasses from sugar cane.

Further examples of suitable carbon sources include, without limitation, peptone, yeast extract, amino acids, other sugars such as arabinose, mannose, glucosamine, maltose, sugar cane, molasses, rum, and the like; salts of organic acids such as acetic acid, fumaric acid, adipic acid, propionic acid, citric acid, gluconic acid, malic acid, pyruvic acid, malonic acid and the like; alcohols such as ethanol, glycerol, and the like; or oil or fat such as soybean oil, rice bran oil, olive oil, corn oil, and sesame oil. The amount of the carbon source added varies according to the kind of carbon source and is typically between 1 to 100 grams per liter of medium. The weight fraction of the carbon source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition. Preferably, molasses is contained in the medium as a carbon source, at a concentration of about 2 to 20% (w/v). More preferably, the molasses is at a concentration of about 8 to 12% (w/v).

One or more embodiments may further comprise one or more essential oils. Such natural oils may include, without limitation, African lemon bush (Lippia javanica) oil, anise oil, bay oil, bergamot oil, boronia oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, ylang ylang oil, and combinations thereof.

The compositions may also include additives. Suitable additives include substances known in the art that may support growth, production of specific metabolites by the microorganism, alter pH, enrich for target metabolites, enhance insecticidal effects, and combinations thereof. Exemplary additives include carbon sources, nitrogen sources, inorganic salt, organic acid, growth media, vitamins, minerals, acetic acid, amino acids, and the like.

In one embodiment, the compositions of the present disclosure may further comprise alcohol. Suitable alcohols include any known in the art including, without limitation, methanol, ethanol, n-propanol, allyl alcohol, n-propanol, isopropanol, sec-propanol, n-butanol, sec-butanol, isobutanol, t-butanol, and tert-amyl-alcohol. The weight fraction of the alcohol in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In another embodiment, the compositions of the present disclosure may further comprise acetic acid, carboxylic acid, or other organic acids. Suitable organic acids include any known in the art including, without limitation, lactic acid, formic acid, acetic acid, propionic acid, butanoic acid, isobutyric acid, 3-methyl butanoic acid, methyl acetate ethyl acetate, propyl acetate, butyl acetate, isobutyl acetate, and 2-methyl butyl acetate. In one embodiment, the acetic acid is included by using vinegar. The weight fraction of the acetic acid in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In yet another embodiment, the compositions of the present disclosure may further comprise any insect repellant, insect-deterrent, or insecticidal composition (collectively “insect repellents”) known in the art. Such insect repellants include, without limitation, N,N-diethyl-m-toluamide (DEET), N,N-diethyl-benzamide, methyl 2-pyrrolidone-5-carboxylate, N-aryl and N-cycloalkyl neo-alkanamides, N-lower alkyl neoalkanamides, nepetalactone and combinations thereof. In some embodiments, one or more insect repellants of the present disclosure may comprise any essential oil.

The weight fraction of the insect repellents in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions of the invention may be in liquid or dry form. The composition may comprise an aqueous suspension of components. This aqueous suspension may be provided as a concentrated stock solution which is diluted prior to application or as a diluted solution ready-to-use. Also, the composition may be a wettable powder, granules, dust, pellet or colloidal concentrate. Such dry forms may be formulated to dissolve immediately upon wetting or dissolve in a controlled-release, sustained-release, or other time-dependent manner. Also, the composition may be in a dry form that does not depend upon wetting or dissolving to be effective.

The compositions may additionally be provided in a formulation capable of spray. The spray may be a liquid or an aerosol.

The compositions of the present disclosure may also be formulated in a nutritional composition (e.g. foodstuff, food additive, dietary supplement, or feed additive). For example, the compositions may be included in food products made using fermentation techniques such as wine, beer, and cheese.

A nutritional composition of the present disclosure may include any of a variety of nutritional agents, which are well known in the art, including vitamins, minerals, essential and non-essential amino acids, carbohydrates, lipids, foodstuffs, dietary supplements, and the like. Thus, the compositions of the present disclosure may include fiber, enzymes and other nutrients. Preferred fibers include, but are not limited to: psyllium, rice bran, oat bran, corn bran, wheat bran, fruit fiber and the like. Dietary or supplementary enzymes such as lactase, amylase, glucanase, catalase and the like can also be included. Vitamins for use in the compositions of the present disclosure include vitamins A, B, C, D, E, folic acid, K, niacin, and the like. Typical vitamins are those, recommended for daily consumption and in the recommended daily amount (RDA).

The compositions of the present disclosure may be formulated in a pharmaceutical composition, where it is mixed with a pharmaceutically acceptable carrier for any type of administration route, selected according to the intended use.

In some embodiments, the combination of the invention may comprise at least one optional excipient. Non-limiting examples of suitable excipients include antioxidants, additives, diluents, binders, fillers, buffering agents, mineral salts, pH modifying agents, disintegrants, dispersing agents, flavoring agents, nutritive agents, oncotic and osmotic agents, stabilizers, preservatives, palatability enhancers and coloring agents. The amount and types of excipients utilized to form the combination may be selected according to known principles of pharmaceutical science.

In one embodiment, the excipient may include at least one diluent. Non-limiting examples of suitable diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lacitol, mannitol, malitol, sorbitol, xylitol, maltodextrin, and trehalose.

In another embodiment, the excipient may comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may include a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may comprise a buffering agent. Representative examples of suitable buffering agents include, but are not limited to, MOPS, HEPES, TAPS, Bicine, Tricine, TES, PIPES, MES, Tris buffers or buffered saline salts (e.g., Tris buffered saline or phosphate buffered saline).

In a further embodiment, the excipient may include a disintegrant. Suitable disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In yet another embodiment, the excipient may include a dispersion enhancer. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In a further embodiment, the excipient may include a lubricant. Non-limiting examples of suitable lubricants include minerals such as talc or silica; and fats such as vegetable stearin, magnesium stearate or stearic acid.

In still another embodiment, it may be desirable to provide a coloring agent. Suitable color additives include, but are not limited to, food, drug and cosmetic colors (FD&C), drug and cosmetic colors (D&C), or external drug and cosmetic colors (Ext. D&C). Such colors may include but not be limited to red, yellow, green, blue, white, black, brown, purple, orange, any shade thereof, and any other color produced according to the above-named color formats.

The weight fraction of the excipient(s) in the combination may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the combination.

The compositions of the present disclosure are stable under various conditions as a liquid or dry form. Preferably, the compositions of the present disclosure are stable at room temperature.

### II. Methods of Formulation

In some embodiments, the present disclosure may provide a method of producing a biofertilizer composition, comprising: (a) obtaining a starting composition comprising a microorganism consortium in culture medium; (b) adding a first additive to the starting composition to produce a first modified composition; (c) adding a second additive to the first modified composition to produce a second modified composition; (d) adding water to the second modified composition to produce a fermented broth; (e) agitating said fermented broth for at least one hour to produce an agitated fermented broth; (f) allowing said agitated broth to settle; and in some embodiments, (g) concentrating said agitated fermented broth to produce a biofertilizer composition product.

The microorganism consortium of the present invention may comprise any combination of microorganism disclosed herein. In some embodiments, the microorganism consortium may comprise at least three microorganisms selected from the group consisting of purple non-sulfur bacteria, chromatiaceae, green sulfur bacteria, colorless sulfur bacteria, filamentous green bacteria, and combinations thereof, wherein at least two microorganisms are selected from the group consisting of Bacillus subtilis, Bifidobacterium animalis, Bifidobacterium bifidum, Bifidobacterium longum, Enterococcus lactis, Streptococcus thermophilus, Lactobacillus acidophilus, Lactobacillus bulgaricus, Lactobacillus casei, Lactobacillus fermentum, Lactobacillus plantarum, and Saccharomyces cerevisiae.

In some embodiments, the microorganism consortium may comprise Bacillus subtilis, a Lactobacillus sp., a Bifidobacterium sp., a Lactococcus sp., Streptococcus thermophilus, a purple non-sulfur bacteria sp., at least two non-pathogenic gram-positive Bacilli bacteria capable of fermentation, and a yeast, and wherein said microorganism consortium is free of detectable non-Bacilli and non-Rhodospirillaceae bacteria.

In some embodiments, the microorganism consortium may comprise Bacillis subtilis, Bacillus coagulans, Lactobacillus acidophilus, Lactobacillus bulgaricus, Lactobacillus casei, Lactobacillus fermentum, Lactobacillus lactis, Lactobacillus parafarraginis, Lactobacillus plantarum, Lactobacillus rapi, Bifidobacterium bifidum, Bifidobacterium longum, Streptococcus thermophilus, Rhodopseudomonas palustris, Rhodobacter sphaeroides, Acetobacter ghanensis, Gluconacetobater diazotrophicus, and Saccharomyces cerevisiae.

In one or more embodiments of the present disclosure, the biofertilizer composition may be prepared partly or entirely within one or more fermentation tanks, or bioreactor as provided in more detail below. In some embodiments, less than ten fermentation tanks may be provided. In some embodiments, less than twenty fermentation tanks may be provided. In some embodiments, more than twenty fermentation tanks may be provided. In some embodiments, one or more fermentation tank may be a primary fermentation tank. In one or more embodiments, one or more fermentation tanks may be a horizontal fermentation tank. In one or more embodiments, one or more fermentation tanks may be a vertical fermentation tank.

In some embodiments, one or more primary fermentation tanks may provide for a maximum capacity of at least 500 liters. In some embodiments, one or more primary fermentation tanks may provide for a maximum capacity of at least 5000 liters. In some embodiments, one or more primary fermentation tanks may hold at least 2006.40 liters. In some embodiments, one or more primary fermentation tanks may hold at least 5639.65 liters. In some embodiments, one or more primary fermentation tanks may hold at least 5678.00 liters. In some embodiments, one or more primary fermentation tanks may hold at least 11440.00 liters.

In some embodiments, one or more horizontal fermentation tanks may provide for a maximum capacity of at least 500 liters. In some embodiments, one or more horizontal fermentation tanks may hold at least 2006.40 liters. In some embodiments, one or more horizontal fermentation tanks may provide for a maximum capacity of at least 5000 liters. In some embodiments, one or more horizontal fermentation tanks may hold at least 5639.65 liters. In some embodiments, one or more horizontal fermentation tanks may hold at least 5678.00 liters. In some embodiments, one or more horizontal fermentation tanks may hold at least 11440.00 liters.

In some embodiments, one or more vertical fermentation tanks may provide for a maximum capacity of at least 500 liters. In some embodiments, one or more vertical fermentation tanks may hold at least 2006.40 liters. In some embodiments, one or more vertical fermentation tanks may provide for a maximum capacity of at least 5000 liters. In some embodiments, one or more vertical fermentation tanks may hold at least 5639.65 liters. In some embodiments, one or more vertical fermentation tanks may hold at least 5678.00 liters. In some embodiments, one or more vertical fermentation tanks may hold at least 11440.00 liters.

In one or more embodiments, the present disclosure may comprise five primary fermentation tanks having capacities of at least 5639.65 liters, two primary fermentation tanks having capacities of at least 11440.00 liters, three horizontal fermentation tanks having capacities of at least 5678.00 liters, and three vertical fermentation tanks having capacities of at least 2006.40 liters.

The present disclosure may provide that a co-cultured microorganism consortium according to any embodiment disclosed or suggested herein may be first provided. Such microorganism consortiums may comprise, without limitation, combinations of one or more strains of one or more of Bacillus subtilis, Bacillus coagulans, Bifidobacterium bifidum, Bifidobacterium longum, Lactobacillus acidophilus, Lactobacillus bulgaricus, Lactobacillus casei, Lactobacillus fermentum, Lactobacillus lactis, Lactobacillus plantarum, Lactobacillus parafarraginis, Lactobacillus rapi, Acetobacter ghanensis, Gluconacetobacter diazotrophicus, Streptococcus thermophilus, Saccharomyces cerevisiae, and at least one purple non-sulfur bacterium. The microorganism consortium may, as provided elsewhere herein, be co-cultured partly or entirely, co-cultured prior to addition to a fermentation tank, co-cultured partly before and partly after addition to a fermentation tank, or co-cultured within a fermentation tank.

In some embodiments, the fermentation tank may comprise a bioreactor. Any and all details applicable to fermentation tanks may apply to one or more bioreactors.

The bioreactor of the present disclosure may, in some embodiments, comprise a batch reactor, a semi-batch reactor, a fed batch reactor, a photobioreactor, a continuous reactor, or another type of bioreactor. The bioreactor's exterior and/or interior may be comprised of stainless steel, copper, plastic, or equivalent material(s). The bioreactor may provide for one or more temperature control elements, one or more aeration elements, one or more agitation elements, one or more baffles, one or more spargers, one or more jackets, one or more scrubbers, and at least one inflow/outflow portal. The bioreactors may comprise an immobilized cell bioreactor, such as a large-scale immobilized cell bioreactor. The bioreactor(s) may be adiabatic or non-adiabatic. The bioreactor may comprise a gas-phase bioreactor, which may be a one-stage, two-stage, or three- or more stage gas-phase bioreactor. The bioreactor may comprise a chemostat or may support chemostatic activity or usage. The bioreactor may comprise a continuous stirred tank reactor. The bioreactor may comprise a plug flow reactor. The bioreactor may be pressurized. When two or more bioreactors are used, the bioreactors may be connected in series, in parallel, or both in series and in parallel. The bioreactor may be used in a fed-batch manner or for semi-continuous fermenter operation. The bioreactor may comprise a moving bed biofilm reactor, packed-bed, a fibrous-bed, or a membrane bioreactor.

In some embodiments, the temperature control element comprises a water jacket inside the bioreactor through which water may flow changing the temperature of the tank and thereby reducing or increasing the temperature of the bioreactor's contents. The temperature control element may comprise one or more coils that may surround the bioreactor's tank, be placed underneath the tank's base, around the top of the tank, or at one or more location.

The bioreactor agitation elements may, in some embodiments, comprise a mixer mounted to a bioreactor top element, a circulation system wherein the contents of the bioreactor are siphoned off from the bottom of the bioreactor and reintroduced at the top of the bioreactor's interior, shaking the bioreactor, shaking the contents of the bioreactor on a platform within the bioreactor, or other known methods of agitation. The agitation element may comprise a gas dispersing agitator.

Additionally, in some embodiments, the agitation element may comprise, in some embodiments, one or more anchor agitator, propeller agitator, radial propeller agitator, turbine agitator, umbrella agitator, paddle(s) agitator, flat blade agitator, ruvastar cyclo agitator, spiral propeller blade agitator, high shear homogenizer, open blade agitator, or other agitation element located inside a bioreactor. In some embodiments, agitation may comprise cycling the fermented broth inside a bioreactor, or by passing air upwards through a fermented broth inside a bioreactor.

The bioreactor's scrubber may comprise a wet scrubbing system, a dry scrubbing system, or both. The present disclosure's outgassing apparatus may comprise a scrubber or scrubbing system.

In some embodiments, the starting composition, first modified solution, second modified solution, fermented broth, agitated fermented broth, or other element of the present disclosure having a microorganism consortium (each a “fermentable element”) may be fermented for a time prior to its insertion into a bioreactor or before its combination with one or more elements.

In some embodiments, the starting composition may be fermented for about 1, 2, 3, 4, 5, 6, 7, 8, 9, 10, 11, 12, 13, 14, 15, 16, 17, 18, 19, 20, 21, 22, 23, 24, 25, or more days. Preferably, the starting composition is fermented for at least about 15 to about 23 days. More preferably, the starting composition is fermented for at least 21 days. In some embodiments, the fermentation time for the starting composition prior to its insertion into a tank or combination with one or more elements is at least 15 days. The fermentation may take place in a tank, a bioreactor, or any other vessel.

In some embodiments, another fermentable element may be fermented for about 1, 2, 3, 4, 5, 6, 7, 8, 9, 10, 11, 12, 13, 14, 15, 16, 17, 18, 19, 20, 21, 22, 23, 24, 25, or more days. Preferably, the other fermentable element is fermented for at least about 15 to about 23 days. More preferably, the other fermentable element is fermented for at least 21 days. In some embodiments, the fermentation time for the other fermentable element prior to its insertion into a tank or combination with one or more elements is at least 15 days. The fermentation may take place in a tank, a bioreactor, or any other vessel.

The present disclosure may provide that one or more volumes of the starting composition be added to one or more primary fermentation tanks. Without limitation, less than 10 liters, less than 500 liters, less than 1000 liters, or more than 1000 liters of the starting composition may be added to one or more primary tanks. In some embodiments, at least 625.2 liters of the starting composition may be added to one or more primary tanks. Relatedly, and without limitation, less than 10 gallons, less than 500 gallons, less than 1000 gallons, or more than 1000 gallons of the starting composition may be added to one or more primary tanks. In some embodiments, at least 165.2 gallons of the starting composition may be added to one or more primary tanks. Moreover, and without limitation, less than 10 kg, less than 500 kg, less than 1000 kg, or more than 1000 kg of the starting composition may be added to one or more primary tanks. In some embodiments, at least 625.2 kg of the starting composition may be added to one or more primary tanks. The starting composition may comprise less than 10%, less than 25%, less than 50%, less than 75%, less than 95%, or less than 99.9% of the total in-tank solution. In some embodiments, the starting composition comprises 60% of the total in-tank solution.

Next, one or more volumes of a first additive may be added to the starting composition in one or more primary fermentation tanks. In some embodiments, the first additive is an organic acid chelating agent. The first additive may be added to the starting composition at an amount less than 10 liters, less than 500 liters, less than 1000 liters, or more than 1000 liters in the one or more primary tanks. In some embodiments, at least 104.2 liters of the first additive may be added to the starting composition in the one or more primary tanks. Relatedly, and without limitation, less than 10 gallons, less than 500 gallons, less than 1000 gallons, or more than 1000 gallons of the first additive may be added to the starting composition in the one or more primary tanks. In some embodiments, at least 27.5 gallons of the first additive may be added to the starting composition in the one or more primary tanks. Moreover, and without limitation, less than 10 kg, less than 500 kg, less than 1000 kg, or more than 1000 kg of the first additive may be added to the starting composition in the one or more primary tanks. In some embodiments, at least 104.2 kg of the first additive may be added to the starting composition in the one or more primary tanks. The first additive may comprise less than 10%, less than 25%, less than 50%, less than 75%, less than 95%, or less than 99.9% of the total in-tank solution. In some embodiments, the first additive comprises 10% of the total in-tank solution. The product of the starting composition and the first additive may comprise the first modified composition.

In some embodiments, following the preceding steps, or in any order, one or more volumes of a second additive may be added to the first modified composition in one or more primary fermentation tanks. In some embodiments, the second additive is a composition comprising one or more of an amino acid, a peptide, a chelated metal, and a salt. The second additive may be added to the first modified composition at an amount less than 10 liters, less than 500 liters, less than 1000 liters, or more than 1000 liters in the one or more primary tanks. In some embodiments, at least 104.2 liters of the second additive may be added to the first modified composition in the one or more primary tanks. Relatedly, and without limitation, less than 10 gallons, less than 500 gallons, less than 1000 gallons, or more than 1000 gallons of the second additive may be added to the first modified composition in the one or more primary tanks. In some embodiments, at least 27.5 gallons of the second additive may be added to the first modified composition in the one or more primary tanks. Moreover, and without limitation, less than 10 kg, less than 500 kg, less than 1000 kg, or more than 1000 kg of the second additive may be added to the first modified composition in the one or more primary tanks. In some embodiments, at least 104.2 kg of the second additive may be added to the first modified composition in the one or more primary tanks. The second additive may comprise less than 10%, less than 25%, less than 50%, less than 75%, less than 95%, or less than 99.9% of the total in-tank solution. In some embodiments, the second additive comprises 10% of the total in-tank solution. The product of the first modified composition and the second additive may comprise the second modified solution.

Water may be added to the second modified composition at an amount less than 10 liters, less than 500 liters, less than 1000 liters, or more than 1000 liters in the one or more primary tanks. In some embodiments, at least 208.4 liters of water may be added to the second modified composition in the one or more primary tanks. Relatedly, and without limitation, less than 10 gallons, less than 500 gallons, less than 1000 gallons, or more than 1000 gallons of water may be added to the second modified composition in the one or more primary tanks. In some embodiments, at least 55.1 gallons of water may be added to the second modified composition in the one or more primary tanks. Moreover, and without limitation, less than 10 kg, less than 500 kg, less than 1000 kg, or more than 1000 kg of water may be added to the second modified composition in the one or more primary tanks. In some embodiments, at least 208.4 kg of water may be added to the second modified composition in the one or more primary tanks. Water may comprise less than 10%, less than 25%, less than 50%, less than 75%, less than 95%, or less than 99.9% of the total in-tank solution. In some embodiments, water comprises 20% of the total in-tank solution. The product of the second modified composition and water may be termed a fermented broth.

The water may comprise any water whether modified or unmodified. In some embodiments, the water may comprise chlorine-free water. Chlorine-free water (or “dechlorinated” water) may be obtained by exposing the water to air and allowing the chlorine to evaporate, by carbon-filtering the water, or any by other known means of dechlorinating water. In some embodiments, however, the water may comprise filtered water, twice filtered water, treated water, water having additives, filtered sewage, runoff water, greywater, tap water, well water, carbonated water, seawater, lakewater, pondwater, rainwater, any of which may be captured, redirected, modified, or produced by known means.

Following the formation of the fermented broth, or at at step prior to the production of the fermented broth, one or more volumes or amounts of a carbon source may be added to the starting composition, the first modified composition, the second modified composition, or the fermented broth, as applicable.

In some embodiments, the fermented broth may be agitated. In some embodiments, the agitation may be constant. In some embodiments, the agitation may be constant and may last for one minute, less than thirty minutes, less than 1 hour, less than 12 hours, less than 24 hours, or more than 24 hours. In some embodiments, the fermented broth may be constantly agitated for at least 1 hour.

Following agitation, in some embodiments, the biofertilizer composition may be extracted.

In some embodiments, following its agitation, the fermented broth may be concentrated. In some embodiments, the fermented broth may be concentrated before being extracted. In some embodiments, the fermented broth may be concentrated after being extracted.

In some embodiments, the step of concentrating the fermented broth or the biofertilizer composition product may comprise decanting the fermented broth or biofertilizer composition. The decanting step may utilize any techniques or decanting equipment known in the art. In some embodiments, the decanting step may be performed until a suitable product is formed and collected. At any step in the method, including but not limited to one or more fermentation steps, some of all of the starting composition, the first modified composition, the second modified composition, or the fermented broth, as applicable, may be held within a primary fermentation tank, a horizontal fermentation tank, a vertical fermentation tank, or any combination of tanks.

In other embodiments, the step of concentrating the fermented broth or biofertilizer composition may comprise passing the fermented broth or biofertilizer through a gravity filtration apparatus, evaporating the fermented broth or biofertilizer via an evaporator, evaporating the fermented broth or biofertilizer via passive evaporation, or concentration by pervaporative separation of the fermented broth or biofertilizer via a pervaporation system.

At any point or step of the method, any mixture, composition, or product of the present disclosure may undergo a fermentation. For example, the starting composition (previously or concurrently with its presence in one or more fermentation tanks), the first modified composition, the second modified composition, or the fermented broth, as applicable, may undergo one or more additional or alternate fermentations. Each of the one or more fermentations may comprise any amount of time known or anticipated to be useful, such as but not limited to about 1, 2, 3, 4, 5, 6, 7, 8, 9, 10, 11, 12, 13, 14, 15, 16, 17, 18, 19, 20, 21, 22, 23, 24, 25, or more days. Preferably, the composition is fermented for at least about 15 to about 23 days. More preferably, the starting composition, the first modified composition, the second modified composition, or the fermented broth, as applicable, may be fermented for at least 21 days. More preferably, the starting composition, the first modified composition, the second modified composition, or the fermented broth, as applicable, may be fermented for at least 15 days.

The decanted product of the fermented broth, in some embodiments, may be the biofertilizer composition, which generally is present in liquid form.

In some embodiments, the method further comprises the step of removing water from the biofertilizer composition to form a solid biofertilizer composition. In some embodiments, the solid biofertilizer composition may comprise ≤10% water (w/w).

### III. Methods of Use

The compositions disclosed herein are useful in agriculture, environmental remediation, human and animal health, food and/or feed, and as chemical replacements. The present disclosure encompasses methods of benefiting an environment or subject that would benefit from a microorganism composition. The methods may be used to replace chemical compositions, such as insecticides, pesticides, or chemicals. The methods may be used to benefit an environment, such as controlling insect populations, enhancing soil for agriculture purposes, and reducing odor associated with waste. Also, the methods may be used to support or enhance health in a human or animal subject. The methods may be used to treat a subject harboring a condition that would benefit from microorganism-based therapy or that is at risk of developing a condition that would benefit from microorganism-based therapy.

The biofertilizer compositions disclosed herein are useful in agriculture applications, including but not limited to soil enrichment, plant enrichment, and enhancing biodegradation. Methods of utilizing the biofertilizer composition in agricultural methods are also provided.

Methods of utilizing the biofertilizer composition in soil enrichment include applying the biofertilizer composition to the soil to be enriched. The biofertilizer composition may be in liquid or dry form and applied to the soil by methods known in the art. Exemplary methods include spraying, dropping, scattering, and dusting the target soil. Also, the biofertilizer composition may be applied to a water source that feeds the target soil.

In another embodiment, the biofertilizer composition may be used for plant enrichment. Methods of plant enrichment include applying the biofertilizer compositions of the invention to the soil or water source of the plant as described herein. Also, the biofertilizer composition may be added to the water of cut flowers or plants. In another embodiment, seeds may be soaked in a biofertilizer composition of the invention prior to planting. It will be recognized that it may be beneficial to combine any of the methods described herein for soil and plant enrichment.

The one or more products made by the processes disclosed and described herein can be applied using any conventional system for applying liquid or solid to a seed or foliar surface or locus. Most commonly, application by spraying will be found most convenient, but other techniques, including application by tumbling, brush or by rope-wick can be used if desired. For spraying, any conventional atomization method can be used to generate spray droplets, including hydraulic nozzles and rotating disk atomizers. Introduction of the composition into an irrigation system can be used.

For folliar surface or locus applications, the application rate of the composition can be (in grams per square centimeter of plant or leaf surface or millileters per gram of plant or leaf weight) between about 0.01 g/cm2 to about 10.0 g/cm2, between about 0.01 ml/g to about 10.0 ml/g dry weight, between about 0.2 g/cm2 to about 2.0 g/cm2, between about 0.2 ml/g to about 2.0 ml/g, between 0.3 g/cm2 to about 1.5 g/cm2, between 0.3 ml/g to about 1.5 ml/g, between about 0.4 g/cm2 to about 1.0 g/cm2, between about 0.4 g/ml to about 1.0 g/ml, greater than about 0.4 g/cm2 to about 1.0 g/cm2 or greater than between about 0.4 g/ml to about 1.0 g/ml, applied in the soil or as a foliar application to the foliage or the locus of the plant.

The one or more products made by the processes disclosed herein can be applied to a single plant (e.g., a houseplant or garden ornamental) or to an assemblage of plants occupying an area. In some embodiments, the product is applied to an agricultural or horticultural crop, more especially a food crop. A “food crop” herein means a crop grown primarily for human or animal consumption. Methods of use are appropriate both for field use and in protected cultivation, for example, greenhouse use.

The one or more products made by the processes disclosed herein may also be beneficial for gramineous (grass family) crops such as cereal crops, including corn, wheat, barley, oats and rice. The one or more products made by the processes disclosed herein may also be appropriate for non-gramineous crops, including vegetable crops, fruit crops, broad-leaved field crops such as soybeans, seed crops or a crop of any species grown specially to produce seed. The terms “fruit” and “vegetable” herein are used in their agricultural or culinary sense, not in a strict botanical sense; for example, tomatoes, cucumbers and zucchini are considered vegetables for present purposes, although botanically speaking it is the fruit of these crops that is consumed.

Vegetable crops for which the one or more products made by the processes disclosed herein can be found useful include without limitation: leafy and salad vegetables such as amaranth, beet greens, bitterleaf, bok choy, Brussels sprout, cabbage, catsear, celtuce, choukwee, Ceylon spinach, chicory, Chinese mallow, chrysanthemum leaf, corn salad, cress, dandelion, endive, epazote, fat hen, fiddlehead, fluted pumpkin, golden samphire, Good King Henry, ice plant, jambu, kai-lan, kale, komatsuna, kuka, Lagos bologi, land cress, lettuce, lizard's tail, melokhia, mizuna greens, mustard, Chinese cabbage, New Zealand spinach, orache, pea leaf, polk, radicchio, rocket (arugula), samphire, sea beet, seakale, Sierra Leone bologi, soko, sorrel, spinach, summer purslane, Swiss chard, tatsoi, turnip greens, watercress, water spinach, winter purslane and you choy, flowering and fruiting vegetables such as acorn squash, Armenian cucumber, avocado, bell pepper, bitter melon, butternut squash, caigua, Cape gooseberry, cayenne pepper, chayote, chili pepper, cucumber, eggplant (aubergine), globe artichoke, luffa, Malabar gourd, parwal, pattypan squash, perennial cucumber, pumpkin, snake gourd, squash (marrow), sweetcorn, sweet pepper, tinda, tomato, tomatillo, winter melon, West Indian gherkin and zucchini (courgette), podded vegetables (legumes) such as American groundnut, azuki bean, black bean, black-eyed pea, chickpea (garbanzo bean), drumstick, dolichos bean, fava bean (broad bean), French bean, guar, haricot bean, hemp, horse gram, Indian pea, kidney bean, lentil, lima bean, marijuana, moth bean, mung bean, navy bean, okra, pea, peanut (groundnut), pigeon pea, pinto bean, rice bean, runner bean, soybean, tarwi, tepary bean, urad bean, velvet bean, winged bean and yardlong bean, bulb and stem vegetables such as asparagus, cardoon, celeriac, celery, elephant garlic, fennel, garlic, kohlrabi, kurrat, leek, lotus root, nopal, onion, Prussian asparagus, shallot, Welsh onion and wild leek, root and tuber vegetables, such as ahipa, arracacha, bamboo shoot, beetroot, black cumin, burdock, broadleaf arrowhead, camas, canna, carrot, cassava, Chinese artichoke, daikon, earthnut pea, elephant-foot yam, ensete, ginger, gobo, Hamburg parsley, horseradish, Jerusalem artichoke, jicama, parsnip, pignut, plectranthus, potato, prairie turnip, radish, rutabaga (swede), salsify, scorzonera, skirret, sweet potato, taro, ti, tigernut, turnip, ulluco, wasabi, water chestnut, yacon and yam, and herbs, such as angelica, anise, basil, bergamot, caraway, cardamom, chamomile, chives, cilantro, coriander, dill, fennel, ginseng, jasmine, lavender, lemon balm, lemon basil, lemongrass, marjoram, mint, oregano, parsley, poppy, saffron, sage, star anise, tarragon, thyme, turmeric and vanilla.

Fruit crops for which the present disclosure can be found useful include without limitation apple, apricot, banana, blackberry, blackcurrant, blueberry, boysenberry, cantaloupe, cherry, citron, clementine, cranberry, damson, dragonfruit, fig, grape, grapefruit, greengage, gooseberry, guava, honeydew, jackfruit, key lime, kiwifruit, kumquat, lemon, lime, loganberry, longan, loquat, mandarin, mango, mangosteen, melon, muskmelon, orange, papaya, peach, pear, persimmon, pineapple, plantain, plum, pomelo, prickly pear, quince, raspberry, redcurrant, starfruit, strawberry, tangelo, tangerine, tayberry, ugli fruit and watermelon.

Seed crops, for example, may comprise any specialized crops used to produce seed of any plant species. The microorganism consortium of the present disclosure may be found useful in applications direct to, including but not limited to cereals (e.g., barley, corn (maize), millet, oats, rice, rye, sorghum (milo) and wheat), non-gramineous seed crops such as buckwheat, cotton, flaxseed (linseed), mustard, poppy, rapeseed (including canola), safflower, sesame and sunflower.

Other crops, not fitting any of the above categories, for which the present disclosure can be found useful include without limitation sugar beet, sugar cane, hops, cannibis (or hemp or marijuana), and tobacco.

Moreover, the products made by the processes disclosed herein may be useful in any hydroponic growing operation on any growing plant, or on any plant rooted in soil.

Each of the crops listed above has its own particular nutrition and disease protection needs. Further optimization of compositions described herein for particular crops can readily be undertaken by those of skill in the art, based on the present disclosure, without undue experimentation.

Methods of using the products made by the processes disclosed and described herein comprise applying a composition as described herein to a seed, to a foliar surface of a plant, or to a locus of the plant or seed.

In another embodiment, the addition of the biofertilizer composition to one or more wastes may have the effect of enhancing biodegradation of the various wastes. Such wastes include, without limitation, wastewater, runoff, food waste, waste produced by humans or animals, and landfill waste. The biofertilizer composition also has the effect of enhancing composting.

The biofertilizer composition may be provided either dried or in liquid form to a waste product. The biofertilizer composition may be provided in a variety of amounts with respect to the weight of the waste product depending on the waste product. In some embodiments, the biofertilizer composition is provided in an amount ranging from about 0.5 to 95.5 wt % of the total weight of the waste product. In some embodiments, the biofertilizer composition is provided in an amount ranging from about 0.5 to 75 wt % of the total weight of the waste product. In some embodiments, the biofertilizer composition is provided in an amount ranging from about 0.5 to 50 wt % of the total weight of the waste product. In some embodiments, the biofertilizer composition is provided in an amount ranging from about 0.5 to 25 wt % of the total weight of the waste product. In another embodiment, the biofertilizer composition is provided in an amount ranging from about 1 to about 3 wt % of the total weight of the waste product. In another embodiment, the amount of biofertilizer composition provided to the waste is about 2 wt % of the total amount of waste.

The biofertilizer composition may be provided in either dry form, liquid form or through the spray. Methods of treating waste products include without limitation, spraying, dusting, sprinkling, liquid inoculation, misting, fumigating, aerosolizing, and other methods known in the art.

It should be understood that the biofertilizer composition used may be provided in the form of pure concentrate (100% concentration) or a diluted composition with additional excipients in the dosage form (i.e. the amount of active ingredient in the composition is less than or equal to 99.99%, and the remainder consists of inactive excipients). If diluted, the amount of biofertilizer composition dispensed in the various dosage forms may range from about 1 to 30%, more preferably between about 4 to 8%. One of skill in the art will appreciate that the volume of active component added to the composition will need to be adjusted to account for the dilution and to ensure the end composition comprises the appropriate final concentration of biofertilizer composition. One of skill in the art will also appreciate that the various components of the biofertilizer composition may be provided in a variety of dosage forms including, but not limited to liquid solution or suspension, emulsion, aerosol, slow release matrices, and the like.

Typical concentration range of microorganisms administered is 1E+3 to 1E+13 cells per day. Preferably, at least about 1E+6, at least about 1E+7, at least about 1E+8 cells per day are administered. However, it will be appreciated that the number of bacteria to be administered will vary according to a number of parameters including subject's size, type of disorder and severity of symptoms.

The biofertilizer composition disclosed herein are also particularly useful as insecticides for topical or systemic application to an environment. Such environments include, without limitation, field crops, grasses, fruits and vegetables, lawns, trees, ornamental plants, sand, humans, animals, and other environments that may benefit from insecticide application. The compositions may be formulated for preventative or prophylactic application to an area, and may in certain circumstances be applied to pets, livestock, animal bedding, humans, or in and around farm equipment, barns, domiciles, agricultural facilities, industrial facilities, and other areas that would benefit from insecticide application.

The biofertilizer composition are applied to an area or environment of the target insect by conventional methods. Such methods include, without limitation, spraying, dusting, sprinkling, soil soaking, soil injection, seed coating, seedling coating, foliar spraying, aerating, misting, atomizing, fumigating, aerosolizing, and other methods known in the art.

The biofertilizer composition may be used in consecutive or simultaneous application to an environmental site alone or in combination with one or more additional insecticides, pesticides, chemicals, fertilizers, or other compounds. The compositions may also be used in conjunction with other treatments such as fertilizers, weed killers, cryoprotectants, surfactants, detergents, insecticidal soaps, dormant oils, polymers, time-release or biodegradable carrier formulations that permit long-term dosing of a target area following a single application of the formulation. Likewise, the formulations may be prepared into edible “baits” or fashioned into insect “traps” to permit feeding or ingestion by a target insect.

An insect as repelled by the composition of this invention includes any member of a large group of invertebrate animals characterized, in the adult state (non-adult insect states include larvae and pupae), by division of the body into head, thorax, and abdomen, three pairs of legs, and, often, but not always) two pairs of membranous wings. This definition therefore includes but is not limited to a variety of biting insects (e.g., ants, bees, black flies, chiggers, fleas, green head flies, mosquitoes, stable flies, ticks, wasps), wood-boring insects (e.g., termites), noxious insects (e.g., house flies, cockroaches, lice roaches, wood lice), and household pests (e.g., flour and bean beetles, dust mites, moths, silverfish, weevils). The compositions of the present disclosure are effective insect repellents against a wide spectra of common insect pests, such as those mentioned above and also including biting insects, wood-boring insects, noxious insects, and household pests, most particularly mosquitoes, sand flies, stable flies, and ticks. The invention also includes effectiveness against all stages of invertebrate animals including adult, larvae, and pupae stages.

Regardless of the method of application, the amount of the composition is applied at an insecticidally-effective amount, which will vary depending on such factors as, for example, the specific target insects to be controlled, the specific environment, location, plant, crop, or agricultural site to be treated, the environmental conditions, and the method, rate, concentration, stability, and quantity of application of the composition. The formulations may also vary with respect to climatic conditions, environmental considerations, frequency of application, and severity of insect infestation.

The concentration of insecticidal biofertilizer composition which is used for environmental, systemic, topical, or foliar application will vary widely depending upon the nature of the particular formulation, means of application, environmental conditions, and degree of activity. Typically, the insecticidal biofertilizer composition will be present in the applied formulation at a concentration of at least about 1% by weight and may be up to and including about 99% by weight. Dry formulations of the biofertilizer composition may be from about 1% to about 99% or more by weight of the composition, while liquid formulations may generally comprise from about 1% to about 99% or more of the composition by weight. As such, a variety of formulations are preparable, including those formulations that comprise from about 5% to about 95% or more by weight of the composition mix, including those formulations that comprise from about 10% to about 90% or more by weight of the biofertilizer composition. Naturally, formulations may comprise from about 15% to about 85% or more by weight of the composition, and formulations comprising from about 20% to about 80% or more by weight of the biofertilizer composition are also considered to fall within the scope of the present disclosure.

In compositions in which intact microorganisms are included, preparations will generally contain from about 1E+4 to about 1E+8 cells/mg, although in certain embodiments it may be desirable to utilize formulations comprising from about 1E+2 to about 1E+4 cells/mg, or when more concentrated formulations are desired, compositions comprising from about 1E+8 to about 1E+10 or 1E+11 cells/mg may also be formulated.

The insecticidal formulation of the invention may be administered to a particular area or environment in one or more applications as needed, with a typical field application rate per hectare ranging on the order of from about 50 g/hectare to about 500 g/hectare of composition, or alternatively, from about 500 g/hectare to about 1000 g/hectare may be utilized. In certain instances, it may even be desirable to apply the composition to a target area at an application rate of from about 1000 g/hectare to about 5000 g/hectare or more of composition. In fact, all application rates in the range of from about 50 g of composition per hectare to about 10,000 g/hectare are contemplated to be useful in the management, control, and killing of target insect pests using such insecticidal formulations. As such, rates of about 100 g/hectare, about 200 g/hectare, about 300 g/hectare, about 400 g/hectare, about 500 g/hectare, about 600 g/hectare, about 700 g/hectare, about 800 g/hectare, about 900 g/hectare, about 1 kg/hectare, about 1.1 kg/hectare, about 1.2 kg/hectare, about 1.3 kg/hectare, about 1.4 kg/hectare, about 1.5 kg/hectare, about 1.6 kg/hectare, about 1.7 kg/hectare, about 1.8 kg/hectare, about 1.9 kg/hectare, about 2.0 kg/hectare, about 2.5 kg/hectare, about 3.0 kg/hectare, about 3.5 kg/hectare, about 4.0 kg/hectare, about 4.5 kg/hectare, about 6.0 kg/hectare, about 7.0 kg/hectare, about 8.0 kg/hectare, about 8.5 kg/hectare, about 9.0 kg/hectare, and even up to and including about 10.0 kg/hectare or greater of composition may be utilized in certain agricultural, industrial, and domestic applications of the insecticidal formulations described herein.

### IV. Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

For clarity, in some embodiments, the composition of the present disclosure may be referred to herein as a “biofertilizer,” a “microorganism consortium,” a “microorganism consortium,” or simply the “composition.” As well, the terms “microorganism” and “microbes” may at times be used interchangeably herein. All elements provided herein are to be understood as being preceded by the descriptor “at least one,” unless otherwise noted. However, for simplicity, at times the descriptor “at least one” may be omitted. Therefore, any item denoted in the singular or not specifically designated as “at least one” or “one or more” should be understood to be provided by the present disclosure in at least one amount or iteration. For example, the term “first adjuvant” should be understood as “at least one first adjuvant,” and so forth.

The term “about” means within the inclusive range of ±15% of the numerical value it modifies.

As used herein, “administering” is used in its broadest sense to mean contacting a subject, surface, liquid, or environment with a composition of the invention.

The term “agriculturally acceptable” applied to a material or composition herein means not unacceptably damaging or toxic to a plant or its environment, and not unsafe to the user or others that may be exposed to the material when used as described herein.

The term “co-culture” refers to a culture of microorganisms that includes at least two microorganisms of the present disclosure, described herein.

A “foliar surface” herein is typically a leaf surface, but other green parts of plants have surfaces that may permit absorption of active ingredient, including petioles, stipules, stems, bracts, flowerbuds, etc., and for present purposes “foliar surfaces” will be understood to include surfaces of such green parts.

The term “insecticidally-effective amount” refers to an amount of the composition that can bring about death to at least one insect, or to noticeably reduce insect growth, feeding, or normal physiological development. This amount will vary depending on such factors as, for example, the specific target insects to be controlled, the specific environment, location, plant, crop, or agricultural site to be treated, the environmental conditions, and the method, rate, concentration, stability, and quantity of application. The formulations may also vary with respect to climatic conditions, environmental considerations, frequency of application, and severity of insect infestation.

The term “detectable” as used herein means at or above the limit of detection of a colony-forming unit of particular bacteria, yeast, or other microbes in a microorganism consortium. Methods for determining the presence of (detecting) a particular species or strain of microbe include culture techniques and non-culture techniques. Culture techniques include the use of selective agar media and determination of colony morphology, such as e.g., LAMVAB and Rogosa agar for Lactobacilli ssp., liver-cysteine-lactose and raffinose for Bifidobacterium ssp., heterotrophic plate counting, and the like. Non-culture techniques include e.g., flow cytometry, direct epifluorescent counting, PCR and other DNA-based methods, such as quantitative PCR, and metabolic/chemical methods, such as propridium monoazide PCR or ethidium monoazide PCR. Methods of detection useful in the practice of this invention are described in Catherine Davis, “Enumeration of biofertilizer strains: Review of culture-dependent and alternative techniques to quantify viable bacteria,” Journal of Microbiological Methods, Volume 103, 2014, pp. 9-17; Jackson and Bird, “Comparison of two selective media for the detection and enumeration of Lactobacilli in human faeces,” Journal of Microbiological Methods 51 (2002) 313-321; Lu et al., “Fine Structure of Tibetan Kefir Grains and Their Yeast Distribution, Diversity, and Shift,” PLoS One. 2014; 9(6): e101387; Rachbid et al., “Assessment of the microbial diversity of Brazilian kefir grains by PCR-DGGE and pyrosequencing analysis,” Food Microbiology, Volume 31, Issue 2, September 2012, Pages 215-221; Furet et al., “Molecular quantification of lactic acid bacteria in fermented milk products using real-time quantitative PCR,” International Journal of Food Microbiology, Volume 97, Issue 2,15 Dec. 2004, Pages 197-207; and Garcia-Cayuel et al., “Simultaneous detection and enumeration of viable lactic acid bacteria and bifidobacteria in fermented milk by using propidium monoazide and real-time PCR,” International Dairy Journal, Volume 19, Issues 6-7, June-July 2009, Pages 405-409.

The phrase “fermentation medium” may refer to a mixture including at least one microorganism, expression products of the microorganism(s), substances produced by the microorganisms, extracts of the microorganisms, and a culture medium or other elements of a culture medium. In some embodiments, the expression product or substance produced by a microorganism may comprise a gas or organic compound product of the fermentation of a carbohydrate, such as an organic acid like acetic acid, citric acid, gluconic acid, lactic acid, propionic acid, pyruvic acid, or succinic acid, a gas like carbon dioxide, or an alcohol like ethanol. In a preferred embodiment, the expression product or substance produced by a microorganism is lactic acid, ethanol, or a combination of lactic acid and ethanol.

“Fermentation product” refers to both or either organic chemical products of a fermentation reaction (e.g., carbon dioxide, ethanol, lactic acid, propionic acid), and/or the fermented carbon source (e.g., fermented sugar cane molasse, fermented rice bran).

The term “finished product” refers to a mixture including a fermentation product. The finished product may include additional additives. Organic fish fertilizer is a finished product.

A “locus” as used herein is inclusive of a foliar surface and also includes an area in proximity to a plant or the area in which a plurality of seed is or can be sown.

The phrase “non-pathogenic gram-positive Bacilli bacteria” refers to those bacteria that belong to the Bacilli taxonomic class of bacteria that contains two orders, Bacillales and Lactobacillales. The term “gram-positive” is used herein to distinguish the subject Bacilli from the group of gram-negative rod-shaped bacteria that are sometimes referred to as bacilli, including for example Escherichia coli and other coliform bacteria. The term “non-pathogenic” is used to refer to those Bacilli that do not cause disease or harm to a plant or animal, i.e., excluding pathogenic bacteria. For example, certain species of Bacillus (e.g., anthraces), Listeria, Staphylococcus, and Streptococcus are pathogenic and are excluded.

Non-limiting examples of non-pathogenic gram-positive Bacilli bacteria include Bifidobacterium spp., Lactobacillus spp., Lactococcus spp., Carnobacterium spp., Streptococcus spp., and Bacillus spp.

The term “microorganism consortium” refers to a composition that contains a live beneficial or useful microorganism alone, in combination with another microorganism, and/or combined with other ingredients such as e.g., energy sources, pre-biotics, stabilizers, and the like, in culture media. Beneficial microorganisms are generally known in the art and include such bacteria as lactic acid fermenting (obligative and facultative) bacteria, phototrophic bacteria, and non-pathogenic bacilli, as well as fermenting yeast such as the Saccharomycetaceae. Microorganism consortiums are generally known to be useful in ameliorating gut flora, remediating wastewater, treating microbial imbalances in animals and plants, protecting animals, plants, and soil from harmful microbes, and improving food animal and food plant production and yield. Useful biofertilizer microorganisms can be found listed for example in Biofertilizer Bacteria: Fundamentals, Therapy, and Technological Aspects, edited by J. Paulo Sousa e Silva, Ana Cristina Freitas, CRC Press, Apr. 2, 2014; and The European Union Register of Feed Additives pursuant to Regulation (EC) No 1831/2003, Annex I: List of additives, available at https://ec.europa.eu/food/sites/food/files/safety/docs/animal-feed-eu-reg comm_register_feed_additives_1831-03.pdf.

The term “pharmaceutical composition” refers to a preparation of one or more compositions of the invention with additional components such as physiologically suitable carriers and excipients. The purpose of a pharmaceutical composition is to facilitate administration of a composition to a recipient subject.

The term “physiologically acceptable carrier” refers to a carrier or a diluent that does not cause significant irritation to a subject and does not abrogate the biological activity and properties of the administered composition.

The term “excipient” refers to an inert substance added to a pharmaceutical composition to further facilitate administration of a composition. Examples, without limitation, of excipients include calcium carbonate, calcium phosphate, various sugars and types of starch, cellulose derivatives, gelatin, vegetable oils and polyethylene glycols. Techniques for formulation and administration of pharmaceutical compositions are known in the art and may be found in “Remington's Pharmaceutical Sciences,” Mack Publishing Co., Easton, Pa., latest edition, which is incorporated herein by reference.

The term “seed” as used herein, is not limited to any particular type of seed and can refer to seed from a single plant species, a mixture of seed from multiple plant species, or a seed blend from various strains within a plant species. The disclosed and described compositions can be utilized to treat gymnosperm seed, dicotyledonous angiosperm seed and monocotyledonous angiosperm seed.

The terms “seed coating” or “seed dressing” as used herein refers generally to a coating or matrix formed on at least part of the seed, the coating or matrix containing or comprising the at least one AI. Optional compounds or agents may be included in the seed coating to facilitate the seed coating process or the disintegration/releasing of the at least one AI from the coating, or to prevent excessive dust-off or to add color to the treated seed.

The term “seed treatment” as used herein refers generally to contacting a seed with a compound or composition of matter containing or comprising at least one active ingredient (a.i. or AI). The compound or composition of matter may be in any form suitable to the seed, for example, liquid, gel, emulsion, suspension, dispersion, spray, or powder. Seed treatment is inclusive of seed coating and seed dressing.

As used herein, “subject” refers to a living organism having a central nervous system. In particular, subjects include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific values (e.g., captive or free specimens of endangered species), or mammals which otherwise have value. Suitable subjects also include: mice, rats, dogs, cats, ungulates such as cattle, swine, sheep, horses, and goats, lagomorphs such as rabbits and hares, other rodents, and primates such as monkeys, chimps, and apes. Subjects may be of any age including new born, adolescence, adult, middle age, or elderly.

As various changes could be made in the above compositions and methods without departing from the scope of the invention, it is intended that all matter contained in the above description and in the Examples given below, shall be interpreted as illustrative and not in a limiting sense.

### V. Examples

### Example 1: Microorganism Consortium Formulation

Biofertilizer compositions were made as follows according to Table 1. Purified water was added to a mixing tank. Molasses (Brix 80+5%, pH 5.7+0.5, Sucrose 30+5% or Total Sugar of 75+5%) was added to the water in the mixing tank. The water and molasses were mixed at 30 Hz speed and then SDA-3C, 199.9+0.1 Proof, 95+1% Ethanol, 4.75+0.50% IPA was added to the mix along with vinegar (120+1 Titratable Acidity). Next, mineral powder (0.15% Mg (as MgO), 0.6% Fe, 0.15% P (as P₂O₅), 3.2% K (as K₂O) was added to the mixture. The mixture was pumped into a fermentation tank. A biofertilizer mix (Bacillus subtilis, Bacilus coagulans, Bifidobacterium bifidum, Bifidobacterium longum, Lactobacillus acidophilus, Lactobacillus bulgaricus, Lactobacillus casei, Lactobacillus fermentum, Lactobacillus lactis, Lactobacillus plantarum, Lactobacillus parafarraginis, Lactobacillus rapi, Streptococcus thermophilus, Acetobacter ghanensis, Gluconacetobacter diazotrophicus, Rhodopseudomonas palustris, Rhodopseudomonas sphaeroides, and Saccharomyces cerevisiae) was added into the mixing tank and blended. Then, the biofertilizer mix was pumped into the fermentation tank. The residue remaining in the mixing tank was rinsed using water and pumped into the fermentation tank. The biofertilizer mix was fermented for 21 days at a temperature of 38° C. and a pH below 3.6.

TABLE 1
Formulations

| | | | | | | | | | |
|---|---|---|---|---|---|---|---|---|---|
| Water (L) | 4968.6 | 4831 | 4581.1 | 4407.2 | 4329 | 4038.6 | 4984 | 4989 | 4713.8 |
| Molasses (L) or | 101.9 | 254.8 | 509.6 | 672 | 764.4 | 1019.2 | 101.9 | 101.9 | 101.9 |
| Rice bran (kg) | | | | | | | | | |
| Mineral Powder | 25.5 | 10.2 | 5.1 | 16.8 | 2.6 | 38.2 | 10.1 | 5.1 | 25.5 |
| (kg) | | | | | | | | | |
| Sea Salt(kg) | 25.5 | 10.2 | 5.1 | 16.8 | 2.6 | 38.2 | 10.1 | 5.1 | 25.5 |
| Biofertilizer | 487.3 | 382.2 | 254.8 | 487.3 | 127.4 | 509.6 | 382.2 | 254.8 | 127.4 |
| Mix (kg) | | | | | | | | | |
| Water (L) | 4955.9 | 4066.6 | 4071.7 | 4051.3 | 4038.6 | 4560.9 | 4560.9 | 4560.9 | 4530.9 |
| Molasses (L) or | 101.9 | 1019.2 | 1019.2 | 1019.2 | 1019.2 | 509.6 | 509.6 | 509.6 | 509.6 |
| Rice bran (kg) | | | | | | | | | |
| Mineral Powder | 38.2 | 10.2 | 5.1 | 25.5 | 38.2 | 25.5 | 25.5 | 25.5 | 25.5 |
| (kg) | | | | | | | | | |
| Sea Salt(kg) | 38.2 | 10.2 | 5.1 | 25.5 | 38.2 | 25.5 | 25.5 | 25.5 | 25.5 |
| Biofertilizer | 51 | 382.2 | 254.8 | 127.4 | 51 | 254.8 | 254.8 | 254.8 | 254.8 |
| Mix (kg) | | | | | | | | | |

What is claimed is:

1. A method of producing a biofertilizer composition, comprising:
a) obtaining a starting composition comprising a microorganism consortium in culture medium, wherein said culture medium contains at least one carbon source;
b) adding a first additive to the starting composition to produce a first modified composition;
c) adding a second additive to the first modified composition to produce a second modified composition;
d) adding water to the second modified composition to produce a fermented broth;
e) agitating said fermented broth for at least one hour to produce an agitated fermented broth;
f) allowing said agitated fermented broth to settle; and
g) extracting a biofertilizer composition product.

2. The method of claim 1, wherein said microorganism consortium comprises Bacillus subtilis, a Lactobacillus sp., a Bifidobacterium sp., a Lactococcus sp., Streptococcus thermophilus, a purple non-sulfur bacteria sp., at least two non-pathogenic gram-positive Bacilli bacteria capable of fermentation, and a yeast.

3. The method of claim 1, wherein said microorganism consortium comprises at least two microorganisms selected from the group consisting of Bacillus subtilis, Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus casei, Lactobacillus fermentum, Lactobacillus plantarum, and Saccharomyces cerevisiae.

4. The method of claim 1, further comprising the step of removing water, wherein said biofertilizer composition product comprises ≤10% water (w/w).

5. The method of claim 1, wherein said first additive is selected from the group consisting of fulvic acid, humic acid, propionic acid, citric acid, homo citric acid, ascorbic acid, lactic acid, oxalic acid, vanillic acid, gallic acid, malate, and gluconic acid.

6. The method of claim 1, wherein said second additive comprises a nitrogen source, a potassium source, a chelated metal, and a salt.

7. The method of claim 1, wherein said second additive comprises water soluble nitrogen, water soluble potash, boron, iron, manganese, and zinc.

8. The method of claim 7, wherein said second additive comprises at least 5.0% (w/w) water soluble nitrogen, 8.0% (w/w) soluble potash, 0.6% (w/w) boron, 1.6% (w/w) iron, 1.6% (w/w) manganese, and 1.6% (w/w) zinc.

9. The method of claim 7, wherein said second additive comprises at least 5.0% (w/w) protein hydrolysate, 8.0% (w/w) potassium citrate, 0.6% (w/w) disodium octaborate tetrahydrate, 1.6% (w/w) ferrous sulfate, 1.6% (w/w) manganese sulfate, and 1.6% (w/w) zinc sulfate.

10. The method of claim 1, wherein said carbon source is selected from the group consisting of molasses, rum, starch, glucose, arabinose, mannose, glucosamine, maltose, sugar cane, and glycerol.

11. The method of claim 1, further comprising the step of adding at least one essential oil to the fermented broth, wherein said at least one essential oil is selected from the group consisting of African lemon bush (Lippia javanica) oil, anise oil, bay oil, bergamot oil, boronia oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, and ylang ylang oil.

12. A biofertilizer composition, comprising:
a) a microorganism consortium in culture medium;
b) at least one an organic acid chelating agent;
c) at least one nitrogen source;
d) at least one potassium source;
e) at least one chelated metal;
f) a salt; and
g) dechlorinated water.

13. The biofertilizer composition of claim 12, wherein said microorganism consortium comprises at least three species selected from the group consisting of Bacillis subtilis, Bacillus coagulans, Lactobacillus acidophilus, Lactobacillus bulgaricus, Lactobacillus casei, Lactobacillus fermentum, Lactobacillus lactis, Lactobacillus parafarraginis, Lactobacillus plantarum, Lactobacillus rapi, Bifidobacterium bifidum, Bifidobacterium longum, Streptococcus thermophilus, Rhodopseudomonas palustris, Rhodobacter sphaeroides, Acetobacter ghanensis, Gluconacetobater diazotrophicus, and Saccharomyces cerevisiae.

14. The biofertilizer composition of claim 12, wherein said at least one organic acid chelating agent is selected from the group consisting of fulvic acid, humic acid, propionic acid, citric acid, homo citric acid, ascorbic acid, lactic acid, oxalic acid, vanillic acid, gallic acid, malate, and gluconic acid.

15. The biofertilizer composition of claim 12, wherein said at least one nitrogen source is selected from the group consisting of soybean hydrolysate, pea protein hydrolysate, whey protein hydrolysate, rice grain protein hydrolysate, alfalfa protein hydrolysate, legume seed protein hydrolysate, corn protein hydrolysate, potassium nitrate, ammonium nitrate, and distiller's dried grains with solubles.

16. The biofertilizer composition of claim 12, wherein said at least one potassium source is selected from the group consisting of potassium oxide, potassium citrate, potassium sulfate, monopotassium phosphate, potassium dihydrogen phosphate, potassium nitrate, potassium chloride, and ammonium phosphate.

17. The biofertilizer composition of claim 12, wherein said at least one chelated metal is a chelated form of at least one element selected from the group consisting of iron, manganese, zinc, copper, magnesium, nickel, boron, and molybdenum.

18. The biofertilizer composition of claim 12, wherein said at least one chelated metal is selected from the group consisting of iron EDDHA, manganese EDTA, and zinc EDTA.

19. The biofertilizer composition of claim 12, wherein said at least one salt is selected from the group consisting of boric acid, disodium octaborate tetrahydrate, sodium molybdate, ferrous sulfate, manganese sulfate, zinc sulfate, potassium dihydrogen phosphate, dipotassium hydrogen phosphate, disodium hydrogen phosphate, magnesium sulfate, magnesium chloride, ferric sulfate, ferric chloride, ferrous chloride, manganous sulfate, manganous chloride, zinc chloride, cupric sulfate, calcium chloride, sodium chloride, calcium carbonate, and sodium carbonate.

20. The biofertilizer composition of claim 12, wherein:
a) said microorganism consortium is present at 50-70% (w/w);
b) said first additive is present at 5-15% (w/w);
c) said second additive is present at 5-15% (w/w); and
d) dechlorinated water is present at 10-30% (w/w).
